(19) United States
(12) Patent Application Publication
(10) Pub. No.: US 2009/0155179 A1
(43) Pub. Date: Jun. 18, 2009

(54) KINETIC BIOMARKER FOR QUANTIFICATION OF LYMPHOPROLIFERATION, CLONAL EXPANSION, RECRUITMENT AND TRAFFICKING IN LYMPHOID TISSUES AND OF THE IN VIVO ACTIONS OF ANTIGENS AND MODULATING THEREON

(75) Inventors: Mark K. Hellerstein, Kensington, CA (US); Robert Busch, Berkeley, CA (US)

Correspondence Address: MORGAN, LEWIS & BOCKIUS, LLP, ONE MARKET SPEAR STREET TOWER, SAN FRANCISCO, CA, 94105, US

(73) Assignee: KINEMED, INC., Emeryville, CA (US)

(21) Appl. No.: 12/065,890

(22) PCT Filed: Sep. 6, 2006
(86) PCT No.: PCT/US2006/034689

§ 371 Date: Oct. 30, 2008

Related U.S. Application Data
(60) Provisional application No. 60/714,873, filed on Sep. 6, 2005.

(51) Int. Cl. A61K 49/00; A61P 35/00

(52) U.S. Cl. 424/9.2

(57) ABSTRACT

The methods of the present invention allow for the measurement of proliferation, clonal expansion trafficking and/or recruitment of lymphocytes into lymphoid tissue in an in vivo setting. Proliferation, clonal expansion, recruitment and/or trafficking of lymphocytes are measured by using stable isotopes to label newly synthesized DNA, isolating the newly-labeled DNA, and quantifying enrichment of the isolated DNA with mass spectrometry or other appropriate techniques. Such methods are useful in screening candidate drugs for stimulatory or inhibitory effects on proliferation, clonal expansion, recruitment and/or trafficking of lymphocytes. The methods also allow for the discovery of therapeutic agents in disorders of immune regulation such as HIV infection and for optimizing vaccine efficacy.

### CROSS-REFERENCE TO RELATED APPLICATIONS

This application claims priority to U.S. provisional application No. 60/714,873 filed on Sep. 6, 2005 which is hereby incorporated by reference in its entirety.

### FIELD OF THE INVENTION

The present invention relates to methods for measuring proliferation, clonal expansion, recruitment and/or trafficking of immune cells and changes in these processes in living organisms. More specifically, the methods allow assessment of the proliferation and trafficking and changes in proliferation and trafficking of lymphocytes in the fields of immunology, vaccines, and basic scientific research. The methods allow for the assessment of the actions of antigens and immune modulating agents (e.g., drugs or drug candidates or vaccines) on the clonal expansion, proliferation, recruitment and/or trafficking of said lymphocytes.

### BACKGROUND OF THE INVENTION

Clonal proliferation of antigen-specific lymphocytes is a fundamental mechanism underlying adaptive immunity. Among lymphocytes there exists a great diversity of antigen receptor structures (B cell antigen receptors=surface immunoglobulins; αβ and χδ T cell antigen receptors; many millions of structural variants for each receptor); each structural variant is generated by somatic gene recombination and somatic mutation events, allowing individual clones of lymphocytes each to express antigen receptors of unique specificity for antigens. Cells with receptors specific for a given antigen receive signals when this antigen is bound by the receptor, which trigger activation and differentiation events that may lead to a host of functional outcomes. These outcomes include the death of antigen-specific cells, induction of functional unresponsiveness, proliferation, and the secretion of cytokines, among others. When antigen-driven proliferation of antigen-specific lymphocytes occurs, it is referred to as clonal expansion, since proliferation occurs selectively in those clones of lymphocytes that receive the antigenic stimuli. Clonal expansion has the net effect of increasing the number of cells that can respond when exposed to the antigen a second time—a phenomenon referred to as immunological memory. The expanded cells also respond more sensitively to antigens upon secondary stimulation, and their responses often are qualitatively different than those of naïve cells.

The magnitude of clonal expansion in response to an antigen is critical for the functional outcome of an immune response. Expansion of antigen-specific lymphocytes is a critical component in diseases of the immune system, including rejection of transplanted organs (lymphocytes specific for alloantigens), autoimmunity (lymphocytes aberrantly responding to self antigens), and allergy (lymphocytes responding to environmental antigens). The development of agents that interfere, with varying degrees of selectivity, with these aberrant lymphoproliferative responses has thus been an area of intense interest in pharmaceutical development. In another example of concern in this field, promising agents can trigger unexpected allergic reactions or autoimmunity in humans, which force the costly withdrawal of a significant fraction of candidate drugs at late stages of clinical development. These drug-triggered or drug-specific responses also involve the stimulation of lymphocyte proliferation.

Certain progressive immunodeficiency syndromes, including HIV-1 infection and acquired immunodeficiency syndrome (AIDS), are characterized by chronic or higher than normal levels immune activation. This activation is believed to contribute to the disease progression (inexorable loss of immune function) by several mechanisms. Use of classic immunosuppressive agents is problematic in these settings, however, because of concerns about worsening the underlying immunodeficiency present.

Chronic immune activation has been shown to stimulate several processes that can contribute to progressive immune deficiency, including: depletion of the naïve T-cell pool by recruiting naïve T-cells into the memory/effector pool; depletion of antigen-specific memory/effector T-cells by activation-induced cell death, leading to holes in the T-cell repertoire; altered thymic function and other aspects of long-term lymphocyte homeostasis; fueling of HIV viral replication by provision of target cells (replicating or activated CD4⁺ T-cells); and damage to the architecture of peripheral lymphoid tissue, including lymph node (LN) fibrosis.

Considerable recent research has strengthened the concept that chronic immune activation, which is often observed in infectious conditions and various immunodeficiency disorders, it of itself potentially damaging to immune homeostasis. A pathophysiologic model has emerged wherein a self-perpetuating cycle evolves, consisting of immune deficiency/immune action/worsened immune deficiency/worsened immune activation/etc.

Chronic immune activation may be identified by use of various metrics. One of the most sensitive indices is increased proliferation rates of T-cells, as has been extensively documented in human subjects with HIV/AIDS. Other measures also can be used and are well known in the art.

Therapeutic efforts to reduce immune activation in HIV/AIDS have, however, been unsystematic and generally unimpressive. Classic immunosuppressant drugs such as CyclosporinA act by reducing lymphocyte proliferation in response to antigenic signaling. Most other immunosuppressive drugs are cytotoxic (i.e., inhibit cell division or cause cell death), including cytoxan, prednisone, and others well known in the art). Use of these agents in disorders characterized by immune deficiency has been met with caution and skepticism by clinicians. Small clinical studies with CyclosporinA in HIV-1 infection have been attempted, with ambiguous results and are not currently considered a promising therapeutic avenue in HIV/AIDS therapeutics.

### SUMMARY OF THE INVENTION

The present invention comprises a drug discovery tool and a therapeutic strategy using drugs so discovered. Applicants disclose herein a means of identifying candidate agents that interfere with lymphocyte proliferation, trafficking and recruitment into lymph nodes (LN) and for identifying optimal doses or regimens of such agents.

Applicants also disclose a therapeutic method for slowing the progression of immune deficiency in certain diseases of immune deficiency, by reducing chronic immune activation without cytotoxic activity or other classic immune toxicities. The method comprises administering agents that reduce or prevent the homing of lymphocytes to inductive sites (such as LNs) in the gut or peripheral tissues, or both, to a subject with an incipient or existing immune deficiency syndrome. As a consequence, several processes involved in the progressive loss of immune function can be reduced, including: lymphocyte activation and proliferation; secondary depletion of naïve T-cells; loss of antigen-specific repertoire in the memory/effector T-cell pool; altered lymphocyte homeostasis, including thymic dysfunction; fueling of HIV-1 replication, in HIV-1 infection, by provision of target cells; and damage to lymph node architecture, including fibrosis. Agents that interfere with lymphocyte trafficking need not have cytotoxic actions and are therefore ideal therapeutic candidates for use in immunodeficiency syndromes.

Applicants also disclose herein a method for identifying candidate agents that are modulators of lymphocyte proliferation, clonal expansion, recruitment and trafficking into LNs and a method for discovering candidate agents that improve the efficacy of vaccines and/or that treat progressive immune deficiency syndromes, including HIV/AIDS by modulating lymphocyte proliferation, and/or clonal expansion, and/or recruitment and/or trafficking into LN.

In another aspect, the present invention provides a method for measuring proliferation, clonal expansion, recruitment or trafficking of lymphocytes, comprising: (a) Administering an antigen (immunogen) to an organism (animal or human subject) into a footpad or other anatomic site known in the art (immunization); (b) Administering, during or after immunization, an isotopic label known to enter DNA during replication; (c) Collection of a lymph node (LN) or LNs from an anatomic site draining the immunization site, at a defined time after immunization; (d) Isolation or separation of cells of interest from the LN; (e) Measurement of proliferation rates (kinetics) of cells isolated, based on the incorporation of label into DNA from said cells; (f) Measurement of LN cellularity (cell counts) on cells for which kinetics are measured; (g) Calculation of clonal expansion (local cell proliferation) and recruitment rates of cells into the LN; and (h) Comparing subjects that received a drug to subjects that were not treated (controls), to identify agents that inhibit cell recruitment into LN or clonal expansion in LN.

In one embodiment the immunogen is KLH, DNCB, or other immunogens known in the art.

In another embodiment, the isotope administered is from a list including ²H₂O, ²H-glucose, ³H-thymidine, BrdU, or other tracers known in the art.

In another embodiment, the LN isolated are popliteal LN and the site of immunogen administration is ipsilateral footpad of a rodent.

In yet another embodiment, the cells of interest isolated from the LN include T-cells, CD₄+ T-cells, B-cells, and other immunocytes known in the art.

In the present invention, the animal used is chosen from fish, rats, mice, rabbits, hamsters, dogs, primates and other animals known in the art.

In another aspect of the invention, the means of measuring proliferation rates (kinetics) of cells is by incorporation of deuterium from ²H₂O.

In another aspect of the invention, the method for measuring cellularity (cell counts) is by fluorescence-activated flow cytometry or other techniques known in the art.

In another aspect of the invention the defined time after immunization is 7 days.

In one embodiment, the drugs compared to controls are from classes that includes inhibitors of cell adhesion, inhibitors of homing receptors, antagonists of vascular receptors, or other inhibition strategies known in the art.

In another embodiment the drugs compared to controls are from classes that include inhibitors of antigen presentation or processing or secretion of cytokines by dendritic cells, or other factors known to be involved in recruitment of cells to LN.

In another embodiment, the dose-response curves are generated for the effects of an agent on cell recruitment or clonal expansion.

The present invention also provides a method for measuring the generation of long-lived memory lymphocyte populations in response to antigen/adjuvant preparations, comprising: (a) immunizing one or more vertebrate animals with or without one or more preparations of antigen with or without one or more adjuvants; (b) administering one or more isotope-labeled precursors of de novo DNA synthesis so as to achieve constant isotopic enrichment in the precursor pool for a defined time period; (c) ceasing label administration for a period of time ranging from 7 days to 2 years; (d) isolating and enumerating one or more lymphocyte populations enriched for proliferating cells; (e) quantifying isotope label retention in DNA isolated from said lymphocyte populations; and (f) calculating the cells remaining since the cessation of label from the amount of retained label and the number of cells in said lymphocyte populations.

In one aspect of the invention, wherein the vertebrate animal is an inbred mouse strain.

In another aspect of the invention, the vertebrate animal is an inbred rodent strain with dissociated lymphocyte proliferation and recruitment

In another aspect of the invention, the inbred rodent strain is the Balb/c mouse strain.

In one embodiment, the antigen is a T-dependent protein antigen.

In another embodiment, the protein antigen is the keyhole limpet hemocyanin.

In another embodiment, the isotope-labeled precursor is heavy water or deuterated glucose.

In another embodiment, the heavy water is ²H₂O.

In one embodiment, the immunization is performed by subcutaneous injection of antigen into a site with a known lymph drainage pattern, and the lymphocyte population enriched for proliferating cells is comprised of lymphocytes from the lymph node or nodes draining said injection site, or phenotypic subsets thereof.

In another embodiment, the present invention provide a method for comparing the magnitude and/or life span of lymphoproliferative responses to two or more alternate antigen preparations, comprising the steps of the method as provided herein, applied to different groups of animals immunized, respectively, with the alternate antigen preparations, or sham-immunized with vehicle preparations lacking antigen.

The present invention also provide a method for measuring the ability of one or more adjuvants to enhance lymphoproliferation or formation of stable memory cells, comprising the steps of the method as provided herein, applied to groups of animals immunized with a reporter antigen formulated with or without the adjuvant or adjuvants.

The present invention also provides a method for measuring the immunostimulatory, allergenic, or autoimmunogenic potential of one or more candidate agents by applying the method provided herein to groups of animals immunized with suspensions or solutions of said compounds or agents in a suitable vehicle as the antigen or antigens, and to control groups of animals without vehicle.

The present invention also provides a method for measuring the modulation of lymphoproliferation and survival by one or more candidate agents wherein said candidate agents are putative immunosuppressants or immunomodulatory agents by applying the methods of provided herein to groups of animals treated systemically or locally with said candidate agents, or treated with vehicle.

The present invention also provides a method for treating chronic, progressive immunodeficiency syndromes in humans by administration of inhibitors of cell recruitment to LN, comprising (a) Treatment with agents identified as inhibitors of cell recruitment to LN by the methods provided herein; (b) Treatment of human subjects with early HIV-1 infection or with AIDS (advanced HIV-infection), using said agents so discovered; (c) Treatment for prolonged or brief periods of time, to reduce immune activation and slow the progression of immune deficiency.

In one embodiment, the effects on immune activation in subjects treated are monitored by measurement of lymphocyte proliferation in the subjects, by use of techniques known in the art.

In another embodiment, the effects on cell recruitment into LN are measured through biopsies of LN tissue, by use of methods disclosed herein.

Accordingly, in one aspect, the present invention provides methods for measuring lymphoproliferation in a living system. The methods comprise administering a stable isotope-labeled substrate to the living system for a period of time sufficient for the substrate to be incorporated into at least one precursor molecule of DNA to form one or more labeled deoxyribonucleotides, optimally but not exclusively purine deoxyribonucleotides, which are then incorporated into growing strands of DNA in cells undergoing S-phase of the cell cycle in the living system. Optionally, several administrations for different time periods can also be performed. A first sample is obtained from the living system; again, optionally multiple samples can be obtained. The isotopic content and/or labeling pattern of deoxyribonucleotides derived from said DNA derived from said sample is then quantified.

In one embodiment, the isotopic content and/or labeling pattern of labeled deoxyribonucleotides is compared to the isotopic content and/or labeling pattern of labeled deoxyribonucleotides observed in a control living system, to determine a difference in lymphoproliferation in the living system as compared to a control living system. In some aspects, the deoxyribonucleotide is a purine deoxyribonucleotide and, specifically, is deoxyadenosine.

In a related embodiment, the cell counts of lymphocytes or subpopulations of lymphocytes in the LN are determined concurrently, by methods known in the art and, by comparison to rates of lymphoproliferation, the rates of cell recruitment and/or trafficking into said LN are calculated, as are the rates of clonal expansion of said lymphocyte populations in said LN.

In a further aspect, the invention provides methods that optionally further comprise administering one or more candidate agents to the living system, either before, during or after the administration of the isotope-labeled substrate. Optional embodiments utilize a first determination of lymphoproliferation, clonal expansion, cell recruitment and/or trafficking in the absence of the one or more candidate agents in a subject and a second determination after the administration of the one or more candidate agents. Optional further embodiments utilize the administration of different concentrations of candidate agents.

In an additional aspect, the invention optionally provides more than one administration of isotope-labeled substrate, e.g., multiple discontinuous administrations. The determination of lymphoproliferation, clonal expansion, cell recruitment and/or trafficking can be done for each administration.

The methods of the present invention find use in all stages of the drug discovery, development, and approval process, as well as in diagnosis of conditions associated with alterations in lymphoproliferation, clonal expansion, cell recruitment and/or trafficking.

Alternatively, the methods of the present invention find use in detecting injuries due to exposure to toxic environmental chemicals such as industrial and occupational chemicals, environmental pollutants, pesticides, food additives, cosmetics, and the like.

### BRIEF DESCRIPTION OF THE DRAWINGS

FIG. 1: Model and Experimental Design. A, Model for effects of cell recruitment, clonal expansion, cell recruitment and/or trafficking on lymph node cellularity (c), the fraction of divided cells (f), and absolute lymphoproliferation (abs) in a 7-day study. a, Homeostatic turnover (black cells) of 10%/week in a resting LN containing 100 lymphocytes. b, Antigenic stimulation without net recruitment expands 1 rare naive precursor present initially (orange cell in a), through 3 cell divisions, generating 7 new antigen-specific responder cells (red), causing a 60% increase in f and a 7% increase in cellularity. c, Recruitment doubles LN cellularity and the number of proliferating cells; f does not change, provided that dividing and resting cells are recruited equally. d, Concurrent effects of recruitment and clonal expansion during an immune response, assuming that the two processes contribute independently. B, Experimental design for continuous ²H₂O labeling of dividing cells in PLN following footpad immunization.

FIG. 2: Increased f in LN cell DNA after immunization. (A) Time course. Balb/c mice were immunized (or not) with 20 μg KLH on day 0 and labeled continuously with ²H₂O. Draining LN cells were analyzed for f. Averages of 2-3 animals per group are shown. #, Day 7 data from a separately labeled cohort of animals. (B) Subset analysis, day 7. PLN cells from ²H₂O-labeled Balb/c mice, immunized 7 days earlier with or without KLH, were sorted as indicated. Means and data from individual animals are shown. *, Significant KLH effect on f for B cells (p<0.001) and T cells (p=0.006; t test), reproduced in 2 or more independent attempts. The KLH effect on CD4+ T cell f did not reach statistical significance here, but other attempts consistently showed a significant effect (e.g., 11.3%±2.1% vs. 7.5%±1.3%; p=0.017). (C) Effects of cyclosporin A (CsA) on T-cell f. Balb/c mice were immunized with 5 μg KLH or PBS, labeled with ²H₂O for 7 days, and given 25 mg/kg/day CsA or vehicle (5% ethanol) p.o. *, Significant effects of immunization (KLH vs. PBS) in vehicle-treated animals (p<0.001), and of CsA treatment on KLH-stimulated (p<0.001) but not on baseline f (p=0.116; 2-way ANOVA, Holm-Sidak post-test). Data from individual mice, means, and SD are shown. (D) Subset analysis of f in PLN on day 7 after immunization with 0.5 mg DNCB or vehicle (1:1 PBS:DMSO). *, Significant DNCB effect on CD4+ (p<0.001) and CD8+ T cells (p=0.005; t test); each subset was analyzed at least twice.

FIG. 3: Responses of f and PLN cellularity to antigens and irritants. (A-C) Balb/c mice were immunized with 20 μg KLH in PBS or IFA, labeled with ²H₂O until day 7, and PLNs were analyzed for f (A) and cellularity (B). Absolute lymphoproliferation was calculated as (cell counts×f) in (C). In A, the KLH effect on PLN cell f was significant (p<0.001), the IFA effect was not (p=0.164; 2-way ANOVA). In B, independent effects of both KLH (p=0.001) and IFA (p<0.001) were significant (2-way ANOVA, log-transformed data). Similar trends were seen in 2 other experiments with 2-3 animals per group. (D-F) Balb/c mice were immunized on day 0 with 0.5 mg DNCB in 1:1 PBS:DMSO, or sham-immunized, as shown. PLN cell f (D), cellularity (E) (note one PBS-treated animal with atypical cell count), and absolute lymphoproliferation (F) are shown. Data from individual animals, means, and SD are shown. *, The DNCB effect on f (D) and new cells (F) was significant (p <0.001 and p=0.02, respectively, by t test vs. DMSO).

FIG. 4: Subset analysis of the response to KLH and IFA. Mice were immunized with or without KLH in PBS or IFA and labeled with ²H₂O as in FIG. 3. Fold changes in f (day 7) relative to baseline (PBS) are shown for sorted B cells (A) and CD4⁺ T cells (B). Results of 2 independent experiments are shown; baseline values of f were, respectively, 8.02% and 9.84% for B cells and 4.63% and 7.77% for CD4+ T cells. By 2-way ANOVA on pooled data, the KLH effect (p<0.001), but not the IFA effect (p=0.085), on B cells was significant. Both the KLH (p<0.001) and IFA (p=0.001) effects on CD4⁺ T cells were significant and mutually independent (p=0.591 for interaction).

FIG. 5: LN cellularity and f during responses to varying doses of antigen. (A-C) Effect of KLH dose. Balb/c mice were immunized with 0, 2.5, 25, or 250 μg KLH in PBS, labeled with ²H₂O, and analyzed for PLN cellularity (A), f (B; average of duplicates) and absolute lymphoproliferation (C) at day 7. Each point represents an individual animal. (D-F) Effect of DNCB dose. Cell counts (D), f (E), and absolute lymphoproliferation (F) are shown 7 days after immunization with DNCB in PBS:DMSO. Individual data, means, and SD are shown. In D, cell counts at 100 μg are different from vehicle controls (*, p=0.003); counts at 500 μg differ from all other groups (#, p<0.001 vs. 0 and 20 μg, p=0.002 vs. 100 μg dose; 1-way ANOVA, Holm-Sidak post-test). In E, mean f differed significantly between all groups (p<0.05), except that the 100 μg and 500 μg doses were not different (p>0.05; ANOVA on ranks, Student-Newman-Keuls post-test).

FIG. 6: Effects of immunization with both KLH and DNCB on PLN cell dynamics. Balb/c mice received immunizations with 20 μg KLH in PBS, followed by 500 μg DNCB in PBS:DMSO, or both, or appropriate vehicles. In order to minimize effects due to the chemical reactivity of DNCB with reactive side chains on KLH, antigens were administered separately, about 6 hours apart, allowing KLH to be captured, processed, and transported to LNs before any modification by DNCB would occur. Analysis of f (A), cell counts (B), and absolute lymphoproliferation (C) in total PLN was performed at day 7. Data from individual animals, means, and SDs are shown. In (A), the effects of both individual antigens on f were significant (p<0.001 vs. control), but dual immunization was indistinguishable from KLH alone (p=0.64; significant interaction between KLH and DNCB effects, p=0.001). In (B) and (C), KLH (p<0.004) and DNCB (p<0.001) effects were independent (all comparisons by 2-way ANOVA, Holm-Sidak method). Results are representative of 3 independent experiments.

FIG. 7: Effects of immunization with KLH and DNCB on f in CD4⁺ T cells and B cells. The experiment in FIG. 6 was repeated. Sorted CD4⁺ T cells (A, C) and B cells (B, D) from PLN were analyzed for f (A, B) and absolute lymphoproliferation (C, D). Additive effects of KLH and DNCB on f of CD4⁺ T cells, and on absolute lymphoproliferation of B and CD4 cells, were significant (p<0.001 for each; p>0.05 for interactions). In (B), KLH (p<0.001), but not DNCB (p=0.358), significantly increased B-cell f; the KLH effect was significantly less with DNCB present (p=0.009; p=0.012 for interaction; all comparisons by 2-way ANOVA, Holm-Sidak method). Similar results were obtained in two independent experiments.

FIG. 8: Differential drug effects on f and LN cellularity. Balb/c mice were immunized with 5 μg KLH or PBS and treated daily with the indicated drugs (Dex, dexamethasone, 0.3 mg/kg/d in cyclodextrin, p.o.; Rap, rapamycin, 2 mg/kg/d in 5% ethanol, p.o.; Tax, paclitaxel, 10 mg/kg/d in 1:1:5 Cremaphor EL:ethanol:water, i.p.; OHU, hydroxyurea, 500 mg/kg/d in water; i.p.; Vehicle, as for paclitaxel group), with continuous ²H₂O labeling until sacrifice at day 7. Proliferating cells in draining LN cells from KLH-immunized (A) or sham-immunized (B) animals were enumerated as (f×cell count). (C—H) Analysis of LN cellularity (C, E, G) and f (D, F, H) in animals treated with or without Tax (C, D), Rap (E, F), or OHU (G, H). Data from individual animals, means, and SD are shown. *, p<0.05 vs. vehicle control by 1-way ANOVA; (*), not significant, but significant effects (p<0.05) of similar magnitude were observed in multiple follow-up studies.

FIG. 9: Lymphoproliferation in response to KLH in C57BI/6 mice. (A-C) B6 mice were immunized with 2-200 μg KLH, and PLN cellularity (A), f (B), and absolute lymphoproliferation (C) were measured at day 7. Data from individual animals are shown. (D) Time course of f after PBS or KLH immunizations. Means and SD are shown for 2-3 animals per group. (E) Analysis of f in sorted B cells and total T cells 7 days after immunization with 200 μg KLH.

FIG. 10 is a schematic diagram showing the drug discovery, development, and approval (DDDA) process using effects on lymphoproliferation (i.e., data collected by the methods of the present invention) as a means for deciding to continue or cease efforts.

FIG. 11 illustrates use of the inventions herein in a drug discovery and development process.

### DETAILED DESCRIPTION OF THE INVENTION

### I. Overview and Introduction of the Invention

A. New Strategies for Reducing Immune Activation:

A strategy for reducing lymphocyte activation without use of cytotoxic agents is highly desirable. In the setting of HIV-1 infection, this therapeutic strategy has recently gained greater credibility, based on published research.

Conversely, the controlled stimulation of antigen-specific lymphoproliferative responses without pathology is the fundamental mechanism of action of vaccines. Therapeutic vaccines must maximize the expansion of effector lymphocytes in a short time span, whereas preventive vaccines must maximize the generation of long-lived memory lymphocytes that provide a persistent pool of primed cells ready to provide protection upon subsequent challenge. Both the initial rate of production of antigen-specific lymphocytes and the longevity of the expanded cell populations thus represent targets for optimization in the field of vaccine development.

In the above fields, there is a significant need for improved in vivo measures of antigen-specific lymphoproliferation for preclinical and clinical research. The great majority of lymphoproliferation studies have been performed in vitro, so that the magnitude of the measured response is limited because the response only indirectly reflects the underlying in vivo cell dynamics.

Furthermore, current methodologies used for proliferation studies in animals have been cumbersome and beset with problems. For example, membrane dye dilution approaches although accurate require isolation and ex vivo chemical labeling of cells of interest with fluorescent dyes, which may alter cellular responsiveness and precludes some experimental designs. In vivo labeling with tritiated nucleoside analogues (commonly ³H-thymidine deoxyribonucleoside, ³H-TdR) or bromodeoxyuridine (BrdU) are of limited value because label incorporation is not uniform per cell, and label retention kinetics in particular are difficult to interpret as a result. Moreover, the labels are hazardous, posing radiation hazards (³H-TdR) or causing myelotoxicity at high doses (BrdU), which are often required to achieve sufficient labeling in vivo. Thus, in practice, they are often used only during the last few hours of an immunization study. Therefore, label incorporation does not fully reflect the degree of lymphoproliferation throughout the course of the study, and may underestimate loss of recently divided cells through apoptosis.

Surrogate markers of proliferation such as DNA content and cell cycle-associated proteins, such as Ki-67 or PCNA, are even less sensitive than labeling methods, since they only identify cells in active cell cycle at the time of sampling. Moreover, they can underestimate differences in proliferation rates, especially in settings of drug-induced cell cycle arrest or elevated apoptosis. For these reasons, studies of cell proliferation have had only a minor role in the in vivo analysis of immune function, as compared to distal measures of immune function such as antibody production, protection from infectious challenge, autoimmune inflammation or tissue damage, or graft rejection.

The present invention solves the above-delineated problems in measuring lymphoproliferation by providing methods that use stable isotopes to label DNA. DNA is uniformly and highly labeled via the de novo synthesis pathway. This method overcomes the difficulties due to non-uniform labeling with nucleotide (salvage) precursors. The stable isotope labels used, e.g., ²H-glucose or heavy water (²H₂O or H₂¹⁸O), are non-toxic to animals and humans, and generally regarded as safe (GRAS) by the US Food and Drug Administration (FDA). Therefore, such an approach is accurate, efficient, and safe in both preclinical and clinical settings.

B. Brief Overview of Invention and Results

How proliferation of antigen-specific lymphocytes and cell recruitment are integrated during polyclonal immune responses remains poorly understood. The Applicants here show that these processes contribute differentially to changes in the fraction of dividing cells (f) and cellularity in antigen-primed lymph nodes. The Applicants determined f from ²H incorporation into DNA of dividing cells after in vivo labeling with ²H₂O. Immunization of Balb/c mice with keyhole limpet hemocyanin (KLH) or 2,4-dinitrochlorobenzene (DNCB), but not injection of incomplete Freund's adjuvant (IFA) or dimethyl sulfoxide, increased f in draining lymph nodes; all stimuli increased cell counts. Lymph node cellularity and f were differentially regulated by antigen dose, addition of IFA, and immunization with two unrelated antigens to increase precursor frequency. In these settings, f reached a plateau, above which absolute lymphoproliferation (f×cellularity) was boosted by increased cell recruitment. Immunosuppressants and antiproliferative agents also differentially modulated cellularity and f. Precursor frequency and calcineurin regulated antigen-stimulated f in CD4+ T cells; plateau values of f in whole lymph nodes reflected differential regulation of T- and B-cell responses. Independent changes in f (reflecting clonal expansion) and cellularity (dominated by cell recruitment) in antigen-stimulated lymph nodes have important implications for vaccine design and discovery of immunomodulatory agents. Please see below for a more detailed description.

C. Introduction of the Invention

Lymph nodes (LNs) provide microenvironments for encounters between antigens and naïve lymphocytes during primary immune responses. Soluble antigen and dendritic cells (DCs) carrying major histocompatibility (MHC)-bound antigen enter LN from inflamed tissues; naïve lymphocytes enter from blood. CD4⁺ T cells within T-cell areas scan dendritic cells for cognate peptide/MHC complexes. When stimulated via their T-cell receptor (TCR), they are retained in the LN, proliferate, and help cognate B cells to proliferate and form germinal centers. Precursor frequency, cell recruitment, antigenic and costimulatory signals, cytokines, and regulatory T cells all shape the primary response. How these factors are integrated, however, remains less well understood. In vivo studies of lymphocyte priming have relied on antigen receptor-transgenic lymphocytes. However, these monoclonal populations may be non-representative in their developmental fate and function; their functional plasticity and proliferative activity depend on the frequency of adoptively transferred cells according to rules that are only beginning to be understood. Lymphocyte recruitment to LNs, by contrast, has been studied mostly using nonspecific stimuli on short time scales; the contribution of cell recruitment to polyclonal responses is not fully understood.

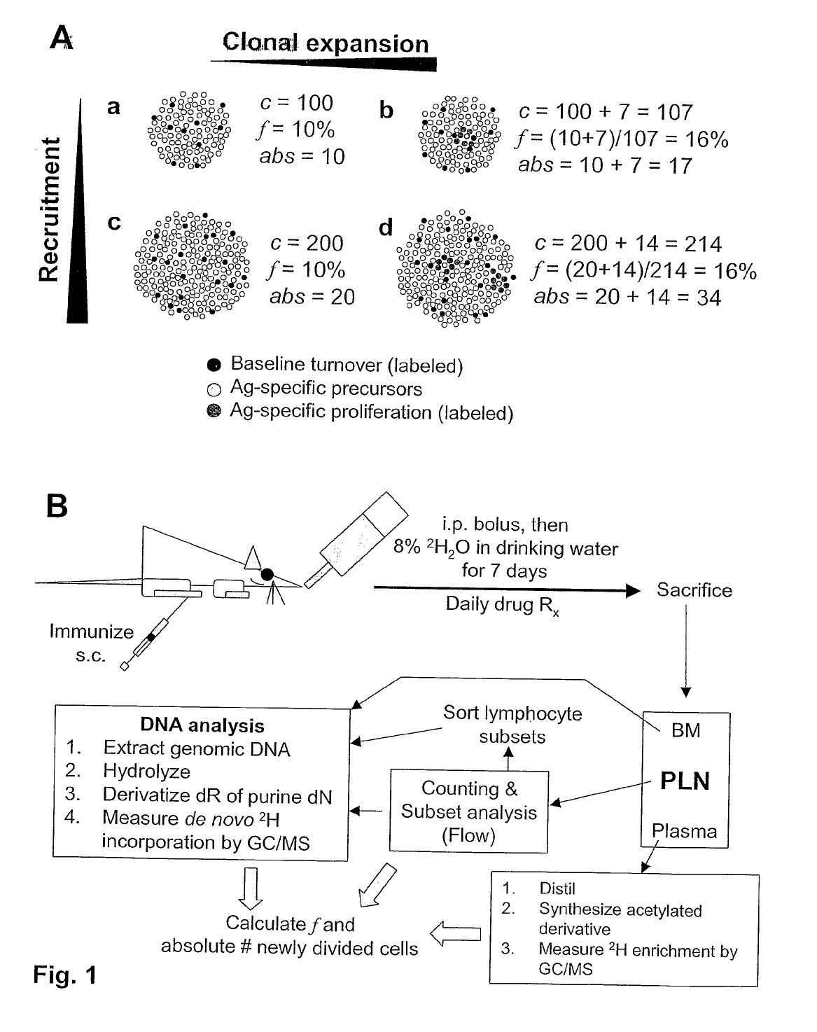

Applicants wished to study the integration of lymphocyte recruitment and antigen-driven proliferation during polyclonal responses in LNs. Applicants reasoned that these processes might differentially affect the percentage and absolute number of dividing cells present (see FIG. 1A). At one extreme, clonal expansion of a few antigen-specific precursors, without recruitment, would increase both the fraction and absolute number of proliferating cells in the LN; the increased cell counts would be entirely due to labeled, recently-divided cells (see FIG. 1A, a vs. b). On the other hand, inflammatory cell recruitment without clonal expansion would increase the absolute number, but not the percentage of dividing cells (see FIG. 1A, a vs. c). In strong immune responses, both an increased fraction of recently divided cells and recruitment of resting and homeostatically proliferating cells contribute to increased cell counts (see FIG. 1A, d).

Accurate quantitation of the fraction (f) of dividing and non-dividing cells in a LN is required to dissect these contributions to lymphocyte dynamics. Commonly-used techniques for measuring proliferation, however, are inadequate to this task. Biosynthetic labeling of dividing cells using pyrimidine nucleoside analogs (e.g., BrdU) may underestimate dividing cells. Incorporation of BrdU into DNA via salvage pathways is inhibited by extracellular nucleosides present in apoptotic microenvironments, such as germinal centers or the thymus. Salvage pathways contribute a variable fraction of newly synthesized DNA. Anti-BrdU staining can vary between similar cell types and can be difficult to detect above background. In vivo, BrdU metabolism can introduce further variation. BrdU is often given as a brief pulse at the end of a study, a design which underestimates total proliferation. When given continuously, BrdU may impair immune responses and affect rapidly turning-over myeloid cells. Thus, BrdU is not well suited for unbiased quantification of dividing and nondividing immune cells. CFSE dilution allows accurate tracking of replicative history, but requires transfer of ex vivo-labeled cells, precluding analysis of primary polyclonal responses.

Applicants have developed an alternative approach, using heavy water (²H₂O) as a non-toxic, non-radioactive label in continuous labeling protocols as more fully described below.

During adaptive immune responses, activated, antigen-bearing dendritic cells and lymphocytes migrate to draining lymph nodes where rare antigen-specific lymphocytes then proliferate, driven by antigen and costimulation. The contributions of lymphocyte migration and clonal expansion to the magnitude of proliferative responses have not yet been determined in normal animals. Here, Applicants have quantified lymphoproliferation by in vivo labeling of de novo synthesized DNA with deuterium (²H), administered orally as ²H₂O.

The absolute number of cells dividing in response to an antigen can be understood as the product of three variables:

1) The effective lymphocyte precursor frequency (PF): The frequency of clonal precursors in a lymphocyte population of interest that are capable of responding to the antigen. This in turn is dictated by the available diversity of the antigen receptor repertoire; by the number of distinct antigenic structures, or antigenic determinants, that may be present in the antigen, having matching antigen receptors in the host's lymphocyte population; and by the affinity of said antigen receptors for said antigenic determinants. The baseline repertoire and the total number of naïve lymphocytes are maintained by a balance between cell death on the one hand and, on the other, the output of primary lymphoid organs (bone marrow, thymus) and homeostatic proliferation.

2) The per-cell efficiency of clonal expansion (CE): The number of successive cell divisions undergone by each precursor cell. This number is influenced by a variety of factors. In primary immune responses, the survival of lymphocytes and the extent of proliferation after antigenic stimulation are controlled by many additional signals that collectively dictate the magnitude of the ensuing clonal expansion. One variable is the local abundance of antigen at the site of lymphoproliferation, for which lymphocytes may compete when antigen is limiting. Another variable is whether the antigen is recognized in a pro-inflammatory or tolerogenic milieu. Inflammation is signaled by proinflammatory cytokines and costimulatory signals provided by accessory cells; tolerance is promoted by anti-inflammatory cytokines and costimulatory molecules. This milieu is regulated in an autocrine and paracrine fashion by lymphocytes themselves and by other cells. For example, B cells are “helped” to secrete antibodies by cytokines and surface interactions provided by CD4⁺ T cells; CD4⁺ T cells in turn are stimulated by dendritic cells; these, in their turn, are stimulated by signs of infection or tissue damage in their tissues of origin, which they recognize as “pathogen-associated molecular patterns” using “pattern recognition receptors”—many of which have recently been found to belong to a family of receptors related to the Drosophila toll gene product (Toll-like receptors, TLRs). Without such proinflammatory stimuli, a tolerogenic milieu is promoted by accessory cells at sites of self-tolerance induction (e.g., the thymus for T cells), by resting or poorly activated dendritic cells in secondary lymphoid organs, etc. Additional counter-regulation can be provided by suppressor or regulatory lymphocytes which, when activated, downregulate antigen responses by other lymphocytes. The phenotypes of suppressor cells are being defined. Secondary immune responses are also subject to regulation, but are more sensitive to antigen and less dependent on the costimulatory milieu than primary responses.

3) The absolute number of lymphocytes that are locally available for antigen encounter (NL). Typically, the initial site of antigen encounter in infections is a secondary lymphoid organ (lymph node or spleen), to which antigens are carried via lymph fluid, either in the fluid phase or by activated dendritic cells entering from inflamed tissue. Naïve and “central memory” lymphocytes constitutively traffic through lymph nodes, entering from blood by extravasation via high endothelial venules, scanning accessory cells in T- and B-cell areas of lymph nodes for cognate antigen presented in a proinflammatory context, exiting via efferent lymph, and re-entering the circulation. At steady state, trafficking through lymph nodes is maintained by specific chemoattractant cytokines (chemokines), which signal to naïve and central memory cells primarily via the chemokine receptor, CCR7. The scanning movements of lymphocytes resemble a random walk of lymphocytes among antigen-presenting and stromal cells. In lymph nodes draining inflamed or infected tissue, increased chemokine secretion by lymphatic endothelia, immigrating dendritic cells and mast cells, and responding lymphocytes causes a considerable additional influx of lymphocytes over baseline. Upon encounter of antigen, lymphocytes cluster around antigen-presenting cells and proliferate locally over several days; a majority dies, but a minority persists as memory cells, declining slowly over months or persisting for life. Another subset differentiates into effector lymphocytes, which emigrate from lymph nodes and can home to sites of inflammation; these migration events are triggered by loss of lymph node homing receptors (CCR7, CD62L) and gradients of chemokines and other chemoattractants directing effector cells to sites of inflammation and infection. Finally, accumulations of infiltrating lymphocytes can be found in chronically inflamed tissues, which establish lymph node-like patterns of chemokine and chemokine receptor expression, and thus a characteristic microarchitecture.

A useful starting point for the analysis of the magnitude of antigen-specific lymphoproliferative responses (LP) is to consider them as the product of each of the above variables:

LP_{Ag}=PF_{Ag}×CE_{Ag}×NL,

where the subscript refers to antigen-specific responses. After a period of antigenic stimulation, the fraction of all cells that has proliferated in response to the antigen is given by

f_{Ag}=PF_{Ag}×CE_{Ag},

where f designates fractional cell proliferation. Of note, in this formulation, the parameter f_{Ag} reflects only the contributions of clonal expansion to the magnitude of the response; passive chemoattraction of cells to a site of inflammation, which occurs regardless of antigenic specificity, does not contribute to f.

Antigen-specific lymphoproliferation is superimposed upon homeostatic proliferation, which is not clonally specific (i.e., PF=1) but occurs at a much lower rate, fₕ, where the subscript h designates homeostatic proliferation. Total lymphoproliferation in an immunized animal thus can be formulated as:

LPₜₗ=LP_{Ag}+LP_{BL}=(PF_{Ag}×CE_{Ag}+fₕ)×NL=(f_{Ag}+fₕ)×NL=f×NL.

The magnitude of clonal expansion in response to an antigen is critical for the functional outcome of an immune response. Expansion of Ag-specific lymphocytes is a critical component in diseases of the immune system, including rejection of transplanted organs (lymphocytes specific for alloantigens), autoimmunity (lymphocytes aberrantly responding to self antigens), allergy (lymphocytes responding to environmental antigens). The development of agents that interfere, to varying degrees of selectivity, with these aberrant lymphoproliferative responses has thus been an area of intense interest in pharmaceutical development. In another example of concern in this field, promising candidate drugs can trigger unexpected allergic reactions or autoimmunity in humans, which force the costly withdrawal of a significant fraction of candidate drugs at late stages of clinical development. These drug-triggered or drug-specific responses also involve the stimulation of lymphocyte proliferation.

Conversely, the controlled stimulation of antigen-specific lymphoproliferative responses without pathology is the fundamental mechanism of action of vaccines. Therapeutic vaccines must maximize the expansion of effector lymphocytes in a short time span, whereas preventive vaccines must maximize the generation of long-lived memory lymphocytes that provide a persistent pool of primed cells ready to provide protection upon subsequent challenge. Both the initial rate of production of antigen-specific lymphocytes and the longevity of the expanded cell populations thus represent targets for optimization in the field of vaccine development.

This approach precisely measures the fraction of cells that have divided during the labeling period (f); the number of proliferating cells is calculated as (f×cell number). Applicants have identified two different mechanisms that increase the number of proliferating B cells and CD4⁺ T cells during primary popliteal lymph node (PLN) responses against keyhole limpet hemocyanin (KLH) in Balb/c mice. Proliferation in response to low doses (0.2-2 μg) of KLH was characterized by an increase in f without a change in PLN cellularity. At higher doses (5-25 μg), or in the presence of incomplete Freund's adjuvant, f reached a plateau, and proliferation was augmented solely by increased PLN cellularity. Antigen-driven lymphoproliferation was inhibited by immunosuppressants. Patterns of lymphoproliferation were strain-dependent. In C57BI/6 mice, constitutive turnover of PLN cells was higher, and the increase in f after KLH priming lower, than in Balb/c mice. Antigen-driven increases in f potentially reflect the frequency of antigen-specific precursors and their replicative capacity. Once maximal local clonal expansion is achieved through antigen and costimulatory signals, immune responses may be further increased by attraction of naïve lymphocytes to draining lymph nodes. The latter process thus becomes a separate target for vaccine optimization. More broadly, stable isotope-based measurements of lymphoproliferation may prove useful for discovering and developing immunomodulatory agents (e.g., candidate drugs, vaccines), enabling mechanistic studies of immunomodulatory agents, and detection of immunotoxicity.

### II. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999), all of which are incorporated by reference for the needed techniques. Furthermore, procedures employing commercially available assay kits and reagents will typically be used according to manufacturer-defined protocols unless otherwise noted. Methods are outlined in U.S. Application No. 2005/0255509, and U.S. Pat. Nos. 7,022,834, 7,001,587, 6,808,875, 6461,806, 6,010,846, 5,910,403, all of which are expressly incorporated by reference and are also useful.

### III. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, Mass isotopomer distribution analysis at eight years: theoretical, analytic and experimental considerations by Hellerstein and Neese (Am J Physiol 276 (Endocrinol Metab. 39) E1146-E1162, 1999, hereby incorporated by reference in its entirety, and in particular for the techniques outlined therein). As appropriate, procedures involving the use of commercially available kits and reagents are generally carried out in accordance with manufacturer defined protocols and/or parameters unless otherwise noted.

By “lymphocyte” herein is meant a type of white blood cell involved in a vertabrate's immune system. There are two broad categories of lymphocytes: the large granular lymphocytes and the small lymphocytes. The large granular lymphocytes are more commonly known as the natural killer cells (NK cells). The small lymphocytes are the T cells and B cells. Lymphocytes play an important and integral part of the body's defenses. Lymphocytes include, but not limited to, B cells, CD3⁺ T cells, CD4⁺ T cells, CD8⁺ T cells, NK T cells, and χδ T cells.

By “lymphocyte population” herein is meant the collective of lymphocytes in a vertebrate's immune system or a part thereof. For example, the lymphocytes in the whole immune system of a vertebrate, in the lymph node, or lymphoid organs of interest.

By “antigen” herein is meant a substance that stimulates an immune response, especially the production of antibodies. Antigens include, but not limited to proteins or polysaccharides. Antigen can be any type of molecule, including small molecules (e.eg. haptens) coupled to a carrier-protein.

By “isotopic enrichment” herein is meant to use methods to enrich the lymphocytes being analyzed that bear an isotopomer of interest.

By “ratio of enrichment” herein is meant the amount of lymphocytes after enrichment verse the amount of lymphocytes before the enrichment.

By “cellularity” herein is meant the physical and chemical properties of cells found within a specific tissue, includes, but not limited to cell counts.

By “long-lived memory lymphocytes” herein is meant a B cell sub-type that are formed following primary infection. When a B cell is activated, by recognizing a specific antigen, it proliferates to form antibody producing plasma cells and long-lived memory cells. The memory B cells are specific for the antigen that first stimulated their production. If this antigen is encountered again, memory B cells can recognize it and quickly proliferate. This forms a new generation of antibody-producing plasma cells.

By “adjuvant” herein is meant an agent which, while not having any specific antigenic effect in itself, may stimulate the immune system, increasing the response to a vaccine.

By “stable memory cell” herein is meant memory B cell that produce antibody against an antigen of interest.

“Molecular flux rates” refers to the rate of synthesis and/or breakdown of molecules within a cell, tissue, or organism. “Molecular flux rates” also refers to a molecule's input into or removal from a pool of molecules, and is therefore synonymous with the flow into and out of said pool of molecules.

“Metabolic pathway” refers to any linked series of two or more biochemical steps in a living system (i.e., a biochemical process), the net result of which is a chemical, spatial or physical transformation of a molecule or molecules. Metabolic pathways are defined by the direction and flow of molecules through the biochemical steps that comprise the pathway. Molecules within metabolic pathways can be of any biochemical class, e.g., including but not limited to lipids, proteins, amino acids, carbohydrates, nucleic acids, polynucleotides, porphyrins, glycosaminoglycans, glycolipids, intermediary metabolites, inorganic minerals, ions, etc.

“Flux rate through a metabolic pathway” refers to the rate of molecular transformations through a defined metabolic pathway. The unit of flux rates through pathways is chemical mass per time (e.g., moles per minute, grams per hour). Flux rate through a pathway optimally refers to the transformation rate from a clearly defined biochemical starting point to a clearly defined biochemical end-point, including all the stages in between in the defined metabolic pathway of interest.

“Isotopes” refer to atoms with the same number of protons and hence of the same element but with different numbers of neutrons (e.g., ¹H vs. ²H or D). The term “isotope” includes “stable isotopes”, e.g. non-radioactive isotopes, as well as “radioactive isotopes”, e.g. those that decay over time.

“Isotopologues” refer to isotopic homologues or molecular species that have identical elemental and chemical compositions but differ in isotopic content (e.g., CH₃NH₂ vs. CH₃NHD in the example above). Isotopologues are defined by their isotopic composition therefore each isotopologue has a unique exact mass but may not have a unique structure. An isotopologue is usually comprised of a family of isotopic isomers (isotopomers) which differ by the location of the isotopes on the molecule (e.g., CH₃NHD and CH₂DNH₂ are the same isotopologue but are different isotopomers).

“Isotope-labeled water” includes water labeled with one or more specific heavy isotopes of either hydrogen or oxygen. Specific examples of isotope-labeled water include ²H₂O and H₂¹⁸O.

“Candidate agent” or “candidate drug” as used herein describes any molecule, e.g., proteins including biotherapeutics including antibodies and enzymes and other biological agents or factors, small organic molecules including new chemical entities, known drugs and drug candidates, polysaccharides, fatty acids, vaccines, nucleic acids, proteins, etc. that can be screened for activity as outlined herein. Candidate agents are evaluated in the present invention for discovering potential therapeutic agents that affect lymphoproliferation and therefore potential disease states, for elucidating toxic effects of agents (e.g. environmental pollutants including industrial chemicals, pesticides, herbicides, etc.), drugs and drug candidates, vaccines, food additives, cosmetics, etc., as well as for elucidating new pathways associated with agents (e.g., research into the side effects of drugs, mechanistic studies of immunomodulating agents including vaccines, etc.). “Candidate agent” and “compound” are used interchangeably herein.

Candidate agents encompass numerous chemical classes. In one embodiment, the candidate agent is an organic molecule, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

“Known drugs” or “known drug agents” or “already-approved drugs” refers to agents (i.e., chemical entities or biological factors) that have been approved for therapeutic use as drugs in human beings or animals in the United States or other jurisdictions. In the context of the present invention, the term “already-approved drug” means a drug having approval for an indication distinct from an indication being tested for by use of the methods disclosed herein. Using immunosuppression and fluoxetine as an example, the methods of the present invention allow one to test fluoxetine, a drug approved by the FDA (and other jurisdictions) for the treatment of depression, for effects on biomarkers of immunosuppression (e.g., inhibition of lymphoproliferation); treating immunosuppression with fluoxetine is an indication not approved by FDA or other jurisdictions. In this manner, one can find new uses (in this example, anti-immunosuppressive effects) for an already-approved drug (in this example, fluoxetine).

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In one embodiment, the candidate agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and norleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations. Peptide inhibitors of enzymes find particular use.

In another embodiment, the candidate agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

In yet another embodiment, the candidate agents are antibodies, a class of proteins. The term “antibody” includes full-length as well antibody fragments, as are known in the art, including Fab Fab2, single chain antibodies (Fv for example), chimeric antibodies, humanized and human antibodies, etc., either produced by the modification of whole antibodies or those synthesized de novo using recombinant DNA technologies, and derivatives thereof.

In still yet another embodiment, the candidate agents are nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook, and peptide nucleic acids. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. etc.

It should be noted in the context of the invention that nucleosides (ribose plus base) and nucleotides (ribose, base and at least one phosphate) are used interchangeably herein unless otherwise noted.

As described above generally for proteins, nucleic acid candidate agents may be naturally occurring nucleic acids, random and/or synthetic nucleic acids. For example, digests of procaryotic or eucaryotic genomes may be used as is outlined above for proteins. In addition, RNAs are included herein.

“Food additive” includes, but is not limited to, organoleptic agents (i.e., those agents conferring flavor, texture, aroma, and color), preservatives such as nitrosamines, nitrosamides, N-nitroso substances and the like, congealants, emulsifiers, dispersants, fumigants, humectants, oxidizing and reducing agents, propellants, sequestrants, solvents, surface-acting agents, surface-finishing agents, synergists, pesticides, chlorinated organic compounds, any chemical ingested by a food animal or taken up by a food plant, and any chemical leaching into (or otherwise finding its way into) food or drink from packaging material. The term is meant to encompass those chemicals which are added into food or drink products at some step in the manufacturing and packaging process, or find their way into food by ingestion by food animals or uptake by food plants, or through microbial byproducts such as endotoxins and exotoxins (pre-formed toxins such as botulinin toxin or aflatoxin), or through the cooking process (such as heterocyclic amines, e.g., 2-amino-3-methyllimidazo[4,5-f]quinolone), or by leaching or some other process from packaging material during manufacturing, packaging, storage, and handling activities.

“Industrial chemical” includes, but is not limited to, volatile organic compounds, semi-volatile organic compounds, cleaners, solvents, thinners, mixers, metallic compounds, metals, organometals, metalloids, substituted and non-substituted aliphatic and acyclic hydrocarbons such as hexane, substituted and non-substituted aromatic hydrocarbons such as benzene and styrene, halogenated hydrocarbons such as vinyl chloride, aminoderivatives and nitroderivatives such as nitrobenzene, glycols and derivatives such as propylene glycol, ketones such as cyclohexanone, aldehydes such as furfural, amides and anhydrides such as acrylamide, phenols, cyanides and nitriles, isocyanates, and pesticides, herbicides, rodenticides, and fungicides.

“Environmental pollutant” includes any chemical not found in nature or chemicals that are found in nature but artificially concentrated to levels exceeding those found in nature (at least found in accessible media in nature). So, for example, environmental pollutants can include any of the non-natural chemicals identified as an occupational or industrial chemical yet found in a non-occupational or industrial setting such as a park, school, or playground. Alternatively, environmental pollutants may comprise naturally occurring chemicals such as lead but at levels exceeding background (for example, lead found in the soil along highways deposited by the exhaust from the burning of leaded gasoline in automobiles). Environmental pollutants may be from a point source such as a factory smokestack or industrial liquid discharge into surface or groundwater, or from a non-point source such as the exhaust from cars traveling along a highway, the diesel exhaust (and all that it contains) from buses traveling along city streets, or pesticides deposited in soil from airborne dust originating in farmlands. As used herein, “environmental contaminant” is synonymous with “environmental pollutant.”

“Living system” includes, but is not limited to, cells (including primary cells), cell lines (including cell lines of healthy and diseased cells), plants and animals, particularly mammals and particularly human. Suitable cells include, but are not limited to, tumor cells of all types (particularly melanoma, myeloid leukemia, carcinomas of the lung, breast, ovaries, colon, kidney, prostate, brain, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, myocytes, fibroblasts, neurons, glial cells, pancreatic cells, intestinal epithelial cells, lymphocytes, erythrocytes, microbial cells and any other cell-type that can be maintained alive and functional in vitro. Microbial and plant cells can also be used.

In one embodiment, the cells may be genetically engineered, that is, contain exogeneous nucleic acid.

The cell may be collected from a multicellular organism and cultured or may be purchased from a commercial source such as the American Type Culture Collection and propagated as a cell line using techniques well known in the art. Suitable cell lines include, but are not limited to, cell lines made from any of the above-mentioned cells, as well as established cell lines such as Suitable cells also include known research cells, including, but not limited to, Jurkat T cells, NIH3T3 cells, CHO, COS, etc. See the ATCC cell line catalog, hereby expressly incorporated by reference. Suitable mammals include, but are not limited to, any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are included within the definition herein. Living systems can either be control systems, which are free from perturbation such as treatment with candidate agents or free of disease or risk of disease, or systems under evaluation. “Living system” includes individual subjects, including human patients.

A “biological sample” encompasses any sample obtained from a living system, including cells, tissues, or an organism. The sample may be solid in nature. The definition also encompasses liquid samples of biological origin, that are accessible from an organism through sampling by minimally invasive or non-invasive approaches (e.g., urine collection, needle aspiration, breast fluid collection from breast ductal lavage, skin scraping, semen collection, vaginal secretion collection, nasal secretion collection, sputum collection, stool collection, and other procedures involving minimal risk, discomfort or effort). The definition also includes samples that have been manipulated in any way after their procurement, such as by treatment with reagents, solubilization, or enrichment for certain components, such as proteins, lipids, carbohydrates, or organic metabolites. The term “biological sample” also encompasses a clinical sample such as biological fluid or tissue sample.

“Biological fluid” refers to, but is not limited to, urine, edema fluid, saliva, lacrimal fluid, inflammatory exudates, synovial fluid, abscess, empyema or other infected fluid, sweat, pulmonary secretions (sputum), seminal fluid, feces, bile, intestinal secretions, vaginal secretions, or any other biological fluid found in spaces external to the body (i.e., luminal or integumentary spaces).

“Exact mass” refers to mass calculated by summing the exact masses of all the isotopes in the formula of a molecule (e.g., 32.04847 for CH₃NHD).

“Nominal mass” refers to the integer mass obtained by rounding the exact mass of a molecule.

“Mass isotopomer” refers to family of isotopic isomers that is grouped on the basis of nominal mass rather than isotopic composition. A mass isotopomer may comprise molecules of different isotopic compositions, unlike an isotopologue (e.g., CH₃NHD, ¹³CH₃NH₂, CH₃¹⁵NH₂ are part of the same mass isotopomer but are different isotopologues). In operational terms, a mass isotopomer is a family of isotopologues that are not resolved by a mass spectrometer. For quadrupole mass spectrometers, this typically means that mass isotopomers are families of isotopologues that share a nominal mass. Thus, the isotopologues CH₃NH₂ and CH₃NHD differ in nominal mass and are distinguished as being different mass isotopomers, but the isotopologues CH₃NHD, CH₂DNH₂, ¹³CH₃NH₂, and CH₃¹⁵NH₂ are all of the same nominal mass and hence are the same mass isotopomers. Each mass isotopomer is therefore typically composed of more than one isotopologue and has more than one exact mass. The distinction between isotopologues and mass isotopomers is useful in practice because all individual isotopologues are not resolved using quadrupole mass spectrometers and may not be resolved even using mass spectrometers that produce higher mass resolution, so that calculations from mass spectrometric data must be performed on the abundances of mass isotopomers rather than isotopologues. The mass isotopomer lowest in mass is represented as M₀; for most organic molecules, this is the species containing all ¹²C, ¹H, ¹⁶O, ¹⁴N, etc. Other mass isotopomers are distinguished by their mass differences from M₀ (M₁, M₂, etc.). For a given mass isotopomer, the location or position of isotopes within the molecule is not specified and may vary (i.e., “positional isotopomers” are not distinguished).

“Mass isotopomer envelope” refers to the set of mass isotopomers comprising the family associated with each molecule or ion fragment monitored.

“Mass isotopomer pattern” refers to a histogram of the abundances of the mass isotopomers of a molecule. Traditionally, the pattern is presented as percent relative abundances where all of the abundances are normalized to that of the most abundant mass isotopomer; the most abundant isotopomer is said to be 100%. The preferred form for applications involving probability analysis, such as mass isotopomer distribution analysis (MIDA), however, is proportion or fractional abundance, where the fraction that each species contributes to the total abundance is used. The term “isotope pattern” may be used synonymously with the term “mass isotopomer pattern.”

“Monoisotopic mass” refers to the exact mass of the molecular species that contains all ¹H, ¹²C, ¹⁴N, ¹⁶O, ³²S, etc. For isotopologues composed of C, H, N, O, P, S, F, Cl, Br, and I, the isotopic composition of the isotopologue with the lowest mass is unique and unambiguous because the most abundant isotopes of these elements are also the lowest in mass. The monoisotopic mass is abbreviated as m₀ and the masses of other mass isotopomers are identified by their mass differences from m₀ (m₁, m₂, etc.).

“Isotopically perturbed” refers to the state of an element or molecule that results from the explicit incorporation of an element or molecule with a distribution of isotopes that differs from the distribution that is most commonly found in nature, whether a naturally less abundant isotope is present in excess (enriched) or in deficit (depleted). Thus the labels of the present invention are isotopically perturbed, as is the DNA into which the labels are incorporated.

“Metabolic precursors” or “precursors” refer to molecules or atoms that enter into molecular end-products of interest through the metabolic processes of the cell or organism (i.e., through biosynthetic, degradative, and/or intermediary metabolic pathways).

“Monomer” refers to a chemical unit that combines during the synthesis of a polymer and which is present two or more times in the polymer.

“Polymer” refers to a molecule synthesized from and containing two or more repeats of a monomer. Polymers may be homopolymers (all monomers identical) or heteropolymers (more than one type of monomer). A “biopolymer” is a polymer synthesized by or in a living system or otherwise associated with a living system.

By “DNA” is meant a polymeric form of deoxyribonucleotides (adenine, guanine, thymine, or cytosine) in double-stranded or single-stranded form, either relaxed or supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. The term captures molecules that include the four bases adenine, guanine, thymine, or cytosine, as well as molecules that include base analogs which are known in the art.

“Isotope labeled substrate” includes any isotope-labeled precursor molecule that is able to be incorporated into DNA (or the deoxyribonucleotide moiety that DNA is comprised of) in a living system. Examples of isotope labeled substrates include, but are not limited to, ²H₂O, ³H₂O, ²H-glucose, ²H-labeled amino acids, ²H-labeled organic molecules, ¹³C-labeled organic molecules, ¹⁴C-labeled organic molecules, ¹³CO₂, ¹⁴CO₂, ¹⁵N-labeled organic molecules and ¹⁵NH₃.

“Deuterated water” refers to water incorporating one or more ²H isotopes.

“Labeled glucose” refers to glucose labeled with one or more ²H isotopes. Specific examples of labeled glucose or 2H-labeled glucose include [6,6-²H₂]glucose, [1-²H₁]glucose, and [1,2,3,4,5,6-²H₇]glucose.

“Administer[ed]” includes a living system exposed to a compound, including candidate agents and labeled substrates, Such exposure can be from, but is not limited to, topical application, oral ingestion, inhalation, subcutaneous injection, intraperitoneal injection, intravenous injection, and intraarterial injection, in animals or other higher organisms. Administration to cells, tissue culture or cell lines can be adding the compound or candidate agent (or combinations of candidate agents or compounds) to the growth media.

By “toxic effect” is meant an adverse response by a living system to a chemical entity or known drug agent. A toxic effect can be comprised of, for example, end-organ toxicity.

“At least partially identified” in the context of drug discovery and development means at least one clinically relevant pharmacological characteristic of a candidate agent has been identified using one or more of the methods of the present invention. This characteristic may be a desirable one, for example, such as the inhibition or modulation of the proliferation, clonal expansion, recruitment and/or trafficking of lymphocytes. Alternatively, a pharmacological characteristic of a candidate agent may be an undesirable one for example, the production of one or more toxic effects such as an increase in lymphoproliferation leading to undesirable outcomes. Of course, a candidate agent can be more than at least partially identified when, for example, when several characteristics have been identified (desirable or undesirable or both) that are sufficient to support a particular milestone decision point along the drug development pathway. Such milestones include, but are not limited to, pre-clinical decisions for in vitro to in vivo transition, pre-IND filing go/no go decision, phase I to phase II transition, phase II to phase III transition, NDA filing, and FDA approval for marketing. Therefore, “at least partially” identified includes the identification of one or more pharmacological characteristics useful in evaluating a a candidate agent in the drug discovery/drug development process as more fully described herein. A pharmacologist or physician or other researcher may evaluate all or a portion of the identified desirable and undesirable characteristics of a candidate agent to establish its therapeutic index. This may be accomplished using procedures well known in the art.

“Manufacturing a candidate agent” in the context of the present invention includes any means, well known to those skilled in the art, employed for the making of a candidate agent product. Manufacturing processes include, but are not limited to, medicinal chemical synthesis (i.e., synthetic organic chemistry), combinatorial chemistry, biotechnology methods such as hybridoma monoclonal antibody production, recombinant DNA technology, and other techniques well known to the skilled artisan. Such a product may be a final drug agent that is marketed for therapeutic use, a component of a combination product that is marketed for therapeutic use, or any intermediate product used in the development of the final drug agent product, whether as part of a combination product or a single product.

By “action” is meant a specific and direct consequence of an intervention such as the administering of a candidate agent.

By “therapeutic action” is meant an effect on a biochemical or molecular process (i.e., the flow of molecules through metabolic pathways or networks) in a manner that is beneficial to the organism. The effect may be responsible for, or contributing in, a causal manner to the initiation, progression, severity, pathology, aggressiveness, grade, activity, disability, mortality, morbidity, disease sub-classification or other underlying pathogenic or pathologic feature of one or more diseases wherein said effect is beneficial to health or otherwise contributes to a desirable outcome (e.g., a desirable clinical outcome).

By “biomarker” is meant a physical, biochemical, or physiologic measurement from or on the organism that represents a true or intended mechanistic target of a compound or a mechanistic event believed to be responsible for, or contributing in, a causal manner to the initiation, progression, severity, pathology, aggressiveness, grade, activity, disability, mortality, morbidity, disease sub-classification or other underlying pathogenic or pathologic feature of one or more diseases. In some embodiments, there may be a correlational effect instead of a causal one. A biomarker may be the target for monitoring the outcome of a therapeutic intervention (i.e., the functional or structural target of a drug agent). As defined herein “biomarker” refers to biochemical processes that are involved in, or are believed to be involved in, the etiology or progression of a disease or disorder. The biochemical process (i.e., the flow of molecules through a targeted metabolic pathway or network) is the focus of analysis (as disclosed herein) since it is the underlying changes of the biochemical process (i.e., molecular flux rates) that may be the significant or authentic target for treatment or diagnostic monitoring of the disease or disorder.

By “evaluate” or “evaluation” or “evaluating,” in the context of the present invention, is meant a process whereby the activity, toxicity, relative potency, potential therapeutic value and/or efficacy, significance, or worth of a candidate agent or combination of candidate agents is determined through appraisal and study, usually by means of comparing experimental outcomes to established standards and/or conditions. The term embraces the concept of providing sufficient information for a decision-maker to make a “go/no go” decision on a candidate agent (or combinations of candidate agents) to proceed further in the drug development process. A “go/no go” decision may be made at any point or milestone in the drug development process including, but not limited to, any stage within pre-clinical development, the pre-clinical to Investigational New Drug (IND) stage, the Phase I to Phase II stage, the Phase II to more advanced phases within Phase II (such as Phase IIb), the Phase II to Phase III stage, the Phase III to the New Drug Application (NDA) or Biologics License Application (BLA) stage, or stages beyond (such as Phase IV or other post-NDA or post-BLA stages). The term also embraces the concept of providing sufficient information to select “best-in-breed” (or “best-of-breed”) in a class of candidate agents.

By “characterize,” “characterizing,” or “characterization,” in the context of the present invention is meant an effort to describe the character or quality of a candidate agent or combination of candidate agents. As used herein, the term is nearly equivalent to “evaluate,” yet lacks the more refined aspects of “evaluate,” in which to “evaluate” a drug includes the ability to make a “go/no go” decision (based on an assessment of therapeutic value) on proceeding with that drug or chemical entity or biological factor through the drug development process.

By “condition” or “medical condition” is meant the physical status of the body as a whole or of one of its parts. The term is usually used to indicate a change from a previous physical or mental status, or an abnormality not recognized by medical authorities as a disease or disorder. Examples of “conditions” or “medical conditions” include, but are not limited to, obesity, cancer, and proliferative diseases.

By “therapeutic effect” is meant any effect elicited by a candidate agent or combination of candidate agents that provide ameliorative or palliative results, or improves, even to the slightest degree, any clinical sign or symptom of a disease or condition.

### IV. Methods of the Invention

A. Summary of the Methods of the Invention

The methods of the present invention generally comprise the following steps:

1) Selection of an organism suitable for study. The organism can be any animal with an adaptive immune system, i.e., any vertebrate animal, including but nor limited to humans, and fish. Rodents may be used, including, but not limited to hamster, rabbits, mice (Mus musculus) and rats (Rattus norvegicus). Inbred strains of rats and mice are especially useful for preclinical toxicology or efficacy screening. Higher mammals, including dogs and nonhuman primates, also may be used. As explained further infra, particularly useful animal strains are those in which lymphoproliferation is uncoupled from lymphocyte recruitment, a situation that enables sensitive measurements of antigen-driven changes in fractional lymphocyte turnover. In one embodiment, Balb/c mice are used when measuring antigen-driven lymphoproliferation in popliteal lymph nodes.

“Living system” includes, but is not limited to, vertebrates, including animals, particularly mammals and particularly human. Included are animals having a variety of disease states, including lymphoproliferation disorders, as well as cancers, e.g., melanoma, myeloid leukemia, carcinomas of the lung, breast, ovaries, colon, kidney, prostate, brain, pancreas and testes.

Suitable mammals include, but are not limited to, any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are included within the definition herein. Living systems can either be control systems, which are free from perturbation such as treatment with candidate agents or free of disease or risk of disease, or systems under evaluation. “Living system” includes individual subjects, including human patients.

Also included in vertebrate are fish, including, but not limited to zebra fish (Danio rerio), fugu fish (Takifugu rubipres), metaka fish (Oryzias latipes)), and tilapia.

In one embodiment, the cells may be genetically engineered, that is, contain exogeneous nucleic acid.

(2) Immunization of animals with an antigen of interest. In one embodiment of the invention, which is useful for the study of immunomodulation, a T-cell-dependent foreign protein antigen, such as keyhole limpet hemocyanin (KLH) and DNCB (2,4-dinitrochlorobenzene), is used, as it readily stimulates both T- and B-cell proliferation in various mouse strains. Other antigens also may be used for specific applications as is well known to those of skill in the art. For instance, in order to measure proliferation of cytotoxic T cells, it may be desirable to immunize with heat-aggregated protein antigens. Other examples include live attenuated or killed preparations of an entire pathogen. The antigen may be introduced either with or without adjuvants known in the art, such as alum, saponin, or incomplete Freund's adjuvant. For measurement of lymphoproliferation in response to allergenic or autoimmunogenic drugs (that is, for detection of immunostimulatory potential, a form of immunotoxicity), the antigen can be comprised of a solution or suspension of drug in a vehicle suitable for injection, such as is commonly used in the popliteal lymph node assay (PLNA), with or without a reporter antigen such as trinitrophenyl-Ficoll (TNP-Ficoll), as is commonly used in the reporter antigen PLNA. Control groups of animals receiving only the injection vehicle are routinely included in these experiments. In principle, any route of immunization used in the art may be employed for this work; one embodiment uses routes of immunization that result in targeted delivery of the antigen to a restricted set of draining lymph nodes (see step (4), below).

(3) Labeling of immunized animals (and sham-immunized controls) with stable isotope precursors of de novo DNA synthesis. In one embodiment of the invention, the precursor is heavy water (²H₂O). In animals, heavy water labeling is conveniently initiated using a bolus injection of 0.9% w/v saline in 99.9 mol % ²H₂O, in an amount sufficient to bring initial ²H enrichment in body water to between 1 and 15 mole percent, and continued for as long as required by administration of ²H₂O in drinking water. For instance, mice are routinely labeled to 5% ²H in body water by bolus administration of 35 ml ²H₂O/0.9% NaCl per kg body weight, and maintained at this enrichment by administration of 8% ²H₂O in drinking water. Other methods for measuring DNA synthesis that are known in the art are also contemplated for use in the present invention.

(4) A method for enriching, anatomically or by cell phenotype, for a defined subset of lymphocytes that includes the antigen-responsive lymphocyte population of interest. This is an important step because the precursor lymphocytes responsive to any given antigen comprise only a small proportion of all lymphocytes, so that without an appropriate enrichment step, antigen-specific proliferation may be difficult to detect over and above background proliferation due to ongoing lymphocyte input from the bone marrow and thymus, homeostatic lymphocyte turnover, and stimulation by environmental antigens. One embodiment of the invention employs anatomic localization of responsive lymphocytes: the route of immunization is chosen so that antigen delivery is targeted into a particular draining lymph node or a limited group of nodes, and antigen-responsive lymphocytes, as a result, respond selectively in these nodes. For example, subcutaneous immunization in the footpad of rodents localizes the antigenic exposure, and hence recruits the responding lymphocytes, selectively to the draining popliteal lymph node. Similarly, subcutaneous immunization at the base of the tail of rodents recruits responsive lymphocytes to inguinal and paraaortic lymph nodes. Other examples will be obvious to one skilled in the art. Alternatively, total lymphocytes may be isolated from these or other secondary lymphoid organs of interest, including the spleen, or from peripheral circulation, and enriched for antigen-responsive cells, based on the expectation that the antigen will stimulate proliferation in particular lymphocyte subsets (such as B cells, CD3⁺ T cells, CD4⁺ T cells, CD8⁺ T cells, NK T cells, χδ cells, identified by expression of appropriate combinations of lineage-specific antigens known in the art); the expression of a phenotype that is selectively expressed (or downregulated) on lymphocytes responding to antigen, such as expression of activation markers; or both. Examples of activation markers include transferrin receptor, HLA-DR (in humans), CD38, CD44, or CD69, CD25, other activation markers known in the art, or combinations thereof. Moreover, enrichment of antigen-responsive cells may be achieved by depletion of lymphocyte subsets that do not show antigen-specific proliferation. The isolation of antigen-specific cells by virtue of their physical interaction with antigens (or antigen oligomers such as peptide/MHC tetramers), or by virtue of their ability to secrete cytokines in response to antigens, can be a useful enrichment criterion in studies aimed at analyzing the long-term persistence and life span of antigen-responsive memory cells after immunization. (It will be a less useful criterion in other settings where the magnitude of a short-term response, as a percentage of a lymphocyte subset of interest, is the relevant experimental parameter and where pure populations of antigen-specific lymphocytes are expected to turn over fully and rapidly). Another way to examine the proliferation of antigen-specific cells involves the use of antigen receptor-transgenic mice, or lymphocytes from such mice transferred into nontransgenic recipients and their isolation by expression of their characteristic clonotype. Selection steps for enrichment or removal of particular cell populations may include any useful method for cell isolation known in the art, for example, fluorescence-activated cell sorting (preparative flow cytometry), counterflow elutriation, density gradient centrifugation, immunomagnetic bead isolation, RosetteSep, panning, or other such methods.

(5) Determination of lymphoproliferation by measuring stable isotope label incorporation into DNA of the lymphocyte subsets of interest, either alone or in combination with enumeration of the cell population of interest, by any experimental method suitable for this purpose. In one embodiment of the invention, the measurement of stable isotope label incorporation into lymphocyte DNA comprises the following steps: (i) extraction of DNA or its release from chromatin without further isolation, hydrolysis of DNA to deoxyribonucleotides, (ii) selective release of deoxyribose from purine deoxyribonucleotides, (iii) derivatization of purine deoxyribose to a volatile derivative (e.g., pentane tetraacetate, pentafluorobenzyl tetraacetyl derivative, or another suitable derivative) suitable for analysis by gas chromatography/mass spectrometry (GC/MS), (iv) GC/MS analysis of said derivative, (v) analysis of the pattern of mass isotopomer abundance of said derivative, and (vi) calculation from said pattern of an excess enrichment value that is a measure of stable isotope incorporation. Specific embodiments of each of these methods have been taught (U.S. Pat. No. 5,910,403, U.S. patent application Ser. No. 10/872,280, herein incorporated by reference in their entirety), and may be used as published, or refinements and modifications of said methods may be used as is well within the capability of those skilled in the art, including but not limited to those disclosed in this application. These measurements yield a measure of the fraction of cells in the isolated cell population that have synthesized DNA during the labeling period (hereinafter referred to as “fractional turnover”, or as “the fraction of new cells”). This number may, by itself, provide information about the dynamics of lymphoproliferation and its modulation by various candidate agents. Additional information may be obtained if the responding cell population is enumerated by an appropriate counting method, such as hemocytometer counting, Coulter counting, flow cytometry calibrated by addition of beads of known concentration to the cell sample, or other methods used in the art for this purpose. The product of the number of cells of interest and their fractional turnover yields the absolute number of cells that have divided during the labeling period.

B. Administering Isotope-Labeled Substrates

As a first step in the methods of the invention, isotope-labeled substrates are administered. These substrates are generally metabolic precursors, e.g., they are taken up within the living system and enzymatically converted; in the present invention, the substrates are converted to deoxynucleosides which are then incorporated into DNA.

1. Administering an Isotope-Labeled Substrate Molecule

Modes of administering the one or more isotope-labeled substrates may vary, depending upon the absorptive properties of the isotope-labeled substrate and the specific biosynthetic pool into which each compound is targeted. Precursors may be administered to whole animals (organisms), including humans, directly for in vivo analysis. In addition, precursors may be administered in vitro to living cells.

Generally, an appropriate mode of administration is one that produces a steady state level of precursor within the biosynthetic pool and/or in a reservoir supplying such a pool for at least a transient period of time. Intravascular or oral routes of administration are commonly used to administer such precursors to organisms, including humans. Other routes of administration, such as subcutaneous or intramuscular administration, optionally when used in conjunction with slow release substrate compositions, are also appropriate. Compositions for injection are generally prepared in sterile pharmaceutical excipients.

As is discussed herein, administration can be done continuously (e.g., up to and/or including the time of sampling) or discontinuously (either as a single dose over time or multiple doses). When discontinuous administration is done, the time of the individual administrations can either be the same or different.

The time of ceasing label administration is varied, e.g., for a period of time ranging from 7 days to 2 years.

a. Labeled Substrates

(1) Isotope Labels

The first step in measuring molecular flux rates involves administering a stable isotope-labeled substrate to a living system. Isotope labels that can be used in accordance with the methods of the present invention include, but are not limited to, ²H, ¹³C, ¹⁵N, ¹⁸O, or other isotopes of elements present in organic systems and capable of labeling DNA precursor molecules. These isotopes, and others, are suitable for all classes of substrates (e.g., precursor molecules) envisioned for use in the present invention. Such precursor molecules include, but are not limited to, nucleic acid precursors.

In one embodiment, the isotope label is ²H.

i. Precursors of Nucleic Acids

Precursors of nucleic acids (i.e., RNA, DNA) are any compounds suitable for incorporation into RNA and/or DNA synthetic pathways. Examples of substrates useful in labeling the deoxyribose ring of DNA include, but are not limited to, [6,6-²H₂]glucose [U-¹³C6]glucose and [2-¹³C₁]glycerol (see U.S. Pat. No. 6,461,806, herein incorporated by reference in its entirety). Labeling of the deoxyribose is superior to labeling of the information-carrying nitrogen bases in DNA because it avoids variable dilution sources. The stable isotope labels are readily detectable by mass spectrometric techniques.

In one embodiment, a stable isotope label is used to label the deoxyribose ring of DNA from glucose, precursors of glucose-6-phosphate or precursors of ribose-5-phosphate. In embodiments where glucose is used as the starting material, suitable labels include, but are not limited to, deuterium-labeled glucose such as [6,6-²H₂]glucose, [1-²H₁]glucose, [3-²H₁]glucose, [²H₇]glucose, and the like; ¹³C-1 labeled glucose such as [1-¹³C₁]glucose, [U-¹³C₆]glucose and the like; and ¹⁸O-labeled glucose such as [1-¹⁸O₂]glucose and the like.

In embodiments where a glucose-6-phosphate precursor or a ribose-5-phosphate precursor is desired, a gluconeogenic precursor or a metabolite capable of being converted to glucose-6-phosphate or ribose-5-phosphate can be used. Gluconeogenic precursors include, but are not limited to, ¹³C-labeled glycerol such as [2-¹³C₁]glycerol and the like, a ¹³C-labeled amino acid, deuterated water (²H₂O) and ¹³C-labeled lactate, alanine, pyruvate, propionate or other non-amino acid precursors for gluconeogenesis. Metabolites which are converted to glucose-6-phosphate or ribose-5-phosphate include, but are not limited to, labeled (²H or ¹³C) hexoses such as [1-²H₁]galactose, [U-¹³C]fructose and the like; labeled (²H or ¹³C) pentoses such as [1-¹³C₁]ribose, [1-²H₁]xylitol and the like, labeled (2H or ¹³C) pentose phosphate pathway metabolites such as [1-²H₁]seduheptalose and the like, and labeled (²H or ¹³C) amino sugars such as [U-¹³C]glucosamine, [1-²H₁]N-acetyl-glucosamine and the like.

The present invention also encompasses stable isotope labels which label purine and pyrimidine bases of DNA through the de novo nucleotide synthesis pathway. Various building blocks for endogenous purine synthesis can be used to label purines and they include, but are not limited to, ¹⁵N-labeled amino acids such as [¹⁵N]glycine, [¹⁵N]glutamine, [¹⁵N]aspartate and the like, ¹³C-labeled precursors such as [1-¹³C₁]glycone, [3-¹³C₁]acetate, [¹³C]HCO₃, [¹³C]methionine and the like, H-labeled precursors such as ²H₂O and O-labeled precursors such as H₂¹⁸O. Also can be used as label are ²H-glucose, ³H-thymidine, and BrdU.

It is understood by those skilled in the art that in addition to the list above, other stable isotope labels which are substrates or precursors for any pathways which result in endogenous labeling of DNA are also encompassed within the scope of the invention. The labels suitable for use in the present invention are generally commercially available or can be synthesized by methods well known in the art.

ii. Water as a Precursor Molecule

Water is a precursor of nucleic acids (see U.S. patent application Ser. No. 10/872,280, incorporated herein by reference). As such, labeled water may serve as a precursor in the methods taught herein (e.g., ²H₂O, H₂¹⁸O).

H₂O availability is probably never limiting for biosynthetic reactions in a cell (because H₂O represents close to 70% of the content of cells, or >35 Molar concentration), but hydrogen and oxygen atoms from H₂O contribute stoichiometrically to many reactions involved in biosynthetic pathways:

e.g.: R—CO—CH2-COOH+NADPH+H₂O→R—CH₂CH₂COOH (fatty acid synthesis).

As a consequence, isotope labels provided in the form of H- or O-isotope-labeled water is incorporated into biological molecules as part of synthetic pathways. Hydrogen incorporation can occur in two ways: into labile positions in a molecule (i.e., rapidly exchangeable, not requiring enzyme catalyzed reactions) or into stable positions (i.e., not rapidly exchangeable, requiring enzyme catalysis). Oxygen incorporation occurs in stable positions.

Some of the hydrogen-incorporating steps from cellular water into C—H bonds in biological molecules only occur during well-defined enzyme-catalyzed steps in the biosynthetic reaction sequence, and are not labile (exchangeable with solvent water in the tissue) once present in the mature end-product molecules. For example, the C—H bonds on glucose are not exchangeable in solution. In contrast, each of the following C—H positions exchanges with body water during reversal of specific enzymatic reactions: C-1 and C-6, in the oxaloacetate/succinate sequence in the Krebs' cycle and in the lactate/pyruvate reaction; C-2, in the glucose-6-phosphate/fructose-6-phosphate reaction; C-3 and C-4, in the glyceraldehyde-3-phosphate/dihydroxyacetone-phosphate reaction; C-5, in the 3-phosphoglycerate/glyceraldehyde-3-phosphate and glucose-6-phosphate/fructose-6-phosphate reactions.

Labeled hydrogen or oxygen atoms from water that are covalently incorporated into specific non-labile positions of a molecule thereby reveals the molecule's “biosynthetic history”—i.e., label incorporation signifies that the molecule was synthesized during the period that isotope-labeled water was present in cellular water.

The labile hydrogens (non-covalently associated or present in exchangeable covalent bonds) in these biological molecules do not reveal the molecule's biosynthetic history. Labile hydrogen atoms can be easily removed by incubation with unlabeled water (H₂O) (i.e., by reversal of the same non-enzymatic exchange reactions through which ²H or ³H was incorporated in the first place.

As a consequence, potentially contaminating hydrogen label that does not reflect biosynthetic history, but is incorporated via non-synthetic exchange reactions, can easily be removed in practice by incubation with natural abundance H₂O.

Analytic methods are available for measuring quantitatively the incorporation of labeled hydrogen atoms into biological molecules (e.g., liquid scintillation counting for ³H; mass spectrometry or NMR spectroscopy for ²H and ¹⁸O). For further discussions on the theory of isotope-labeled water incorporation, see, for example, Jungas R L. Biochemistry. 1968 7:3708-17, incorporated herein by reference.

Labeled water may be readily obtained commercially. For example, ²H₂O may be purchased from Cambridge Isotope Labs (Andover, Mass.). ²H₂O may be administered, for example, as a percent of total body water, e.g., 1% of total body water consumed (e.g., for 3 liters water consumed per day, 30 microliters ²H₂O is consumed).

Relatively high body water enrichments of ²H₂O (e.g., 1-10% of the total body water is labeled) may be achieved relatively inexpensively using the techniques of the invention. This water enrichment is relatively constant and stable as these levels are maintained for weeks or months in humans and in experimental animals without any evidence of toxicity. This finding in a large number of human subjects (>100 people) is contrary to previous concerns about vestibular toxicities at high doses of ²H₂O. One of the Applicants has discovered that as long as rapid changes in body water enrichment are prevented (e.g., by initial administration in small, divided doses), high body water enrichments of ²H₂O can be maintained with no toxicities. For example, the low expense of commercially available ²H₂O allows long-term maintenance of enrichments in the 1-5% range at relatively low expense.

Relatively high and relatively constant body water enrichments for administration of H₂¹⁸O may also be accomplished, since the ¹⁸O isotope is not toxic, and does not present a significant health risk as a result.

Isotope-labeled water may be administered via continuous isotope-labeled water administration, discontinuous isotope-labeled water administration, or after single or multiple administration of isotope-labeled water administration. In continuous isotope-labeled water administration, isotope-labeled water is administered to an individual for a period of time sufficient to maintain relatively constant water enrichments over time in the individual. For continuous methods, labeled water is optimally administered for a period of sufficient duration to achieve a steady state concentration (e.g., 3-8 weeks in humans, 1-2 weeks in rodents).

In discontinuous isotope-labeled water administration, an amount of isotope-labeled water is measured and then administered, one or more times, and then the exposure to isotope-labeled water is discontinued and wash-out of isotope-labeled water from body water pool is allowed to occur. The time course of delabeling may then be monitored. Water is optimally administered for a period of sufficient duration to achieve detectable levels in biological molecules.

Isotope-labeled water may be administered to an individual or tissue or cells in various ways known in the art. For example, isotope-labeled water may be administered orally, parenterally, subcutaneously, intravascularly (e.g., intravenously, intraarterially), or intraperitoneally. Several commercial sources of ²H₂O and H₂¹⁸O are available, including Isotec, Inc. (Miamisburg Ohio, and Cambridge Isotopes, Inc. (Andover, Mass.). The isotopic content of isotope-labeled water that is administered can range from about 0.001% to about 20% and depends upon the analytic sensitivity of the instrument used to measure the isotopic content of the biological molecules. In one embodiment, 4% ²H₂O in drinking water is orally administered. In another embodiment, a human is administered 50 mL of ²H₂O orally.

The living system being administered ²H₂O may be a cell. The cell may be collected from a multicellular organism and cultured or may be purchased from a commercial source such as the American Type Culture Collection and propagated as a cell line using techniques well known in the art. Alternatively, the individual being administered ²H₂O may be any multicellular organism including a mammal such as a rodent or a human.

In variations involving the administering of drugs, drug candidates, drug leads, biological factors, or combinations thereof (i.e., compounds, combinations of compounds, or mixtures of compounds), the individual may be a mammal, such as an experimental animal, including an accepted animal model of disease, or a human. In variations involving the administering of food additives, industrial or occupational chemicals, environmental pollutants, or cosmetics, the individual may be any experimental animal such as, without limitation, a rodent, primate, hamster, guinea pig, dog, or pig.

C. Administration of Candidate Agents

As outlined herein, candidate agents are administered for a variety of reasons. In some embodiments, the candidate agents are evaluated in the present invention for discovering potential therapeutic agents or vaccines that affect lymphoproliferation, clonal expansion, cell recruitment and/or trafficking and therefore potential disease states, for elucidating toxic effects of agents (e.g., environmental pollutants including industrial chemicals, pesticides, herbicides, etc.), drugs and drug candidates, food additives, cosmetics, vaccines, etc., as well as for elucidating new pathways of lymphoproliferation, clonal expansion, cell recruitment and/or trafficking associated with agents and vaccines (e.g., research into the side effects of drugs, mechanism of action studies, etc.). Administration is accomplished in a variety of ways, as is outlined herein. In many cases multiple concentrations and/or exposure times of candidate agents can be done. Administration can be done prior to, during or after the administration of the isotope-labeled substrate.

As outlined herein, the animals are immunized with antigen of interest by exposing to at least one antigen. The animals are also exposed to at least one candidate agent. In one embodiment, the step of immunization is done prior to the step of exposing the animals to the candidate agent. In another embodiment, the step of immunization is done after the step of exposing the animals to the candidate agent. Alternatively, the step of immunization is done simultaneously with the step of exposing the animals to the candidate agent.

D. Obtaining One or More Targeted DNA Molecules of Interest

In practicing the methods of the invention, in one aspect, targeted DNA molecules of interest are obtained from a cell, tissue, or organism according to methods known in the art. DNA molecules of interest may be isolated from a biological sample.

A plurality of DNA molecules of interest may be acquired from the cell, tissue, or organism. The one or more biological samples may be one or more biological fluids. DNA molecules of interest also may be obtained, and optionally partially purified or isolated, from the biological sample using standard biochemical methods known in the art.

The DNA molecules can also be obtained from tissues of a vertebrate, particularly, a tissue of the immune system, including, but not limited to, lymph node.

The frequency of biological sampling can vary depending on different factors. Such factors include, but are not limited to, the nature of the DNA molecules of interest, ease and safety of sampling, synthesis and breakdown/removal rates of the DNA molecules of interest, and the half-life of a compound, vaccine, or candidate agent.

The DNA molecules of interest may also be purified partially, or optionally, isolated, by conventional DNA purification methods and/or other separation methods known to those skilled in the art.

In another embodiment, the DNA molecules of interest may be hydrolyzed or otherwise degraded to form smaller molecules. Hydrolysis methods include any method known in the art, including, but not limited to, chemical hydrolysis (such as acid hydrolysis) and biochemical hydrolysis (such as nuclease degradation). Hydrolysis or degradation may be conducted either before or after purification and/or isolation of the DNA molecules of interest. The DNA molecules of interest may also be partially purified, or optionally, isolated, by conventional DNA purification methods known to those skilled in the art.

E. Analysis

Presently available technologies (static methods) measure only composition, structure, or concentrations of molecules in a cell and do so at one point in time.

1. Mass Spectrometry

Mass spectrometers convert components of a sample into rapidly moving gaseous ions and separate them on the basis of their mass-to-charge ratios. The distributions of isotopes or isotopologues of ions, or ion fragments, may thus be used to measure the isotopic enrichment in one or more DNA molecules of interest.

Generally, mass spectrometers include an ionization means and a mass analyzer. A number of different types of mass analyzers are known in the art. These include, but are not limited to, magnetic sector analyzers, electrostatic analyzers, quadrupoles, ion traps, time of flight mass analyzers, and fourier transform analyzers. In addition, two or more mass analyzers may be coupled (MS/MS) first to separate precursor ions, then to separate and measure gas phase fragment ions.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

When GC/MS is used to measure mass isotopomer abundances of organic molecules, hydrogen-labeled isotope incorporation from labeled water is amplified 3 to 7-fold, depending on the number of hydrogen atoms incorporated into the organic molecule from labeled water.

In one embodiment, isotope enrichments of DNA molecules of interest may be measured directly by mass spectrometry.

In another embodiment, the DNA molecules of interest may be partially purified, or optionally isolated, prior to mass spectral analysis. Furthermore, hydrolysis or degradation products of DNA molecules of interest may be purified.

In another embodiment, isotope enrichments of DNA molecules of interest after hydrolysis of the said DNA molecules of interest are measured by gas chromatography-mass spectrometry.

In each of the above embodiments the biosynthesis rate of the biological molecule (i.e., DNA molecule of interest) can be calculated by application of the precursor-product relationship (discussed further, infra) using either labeled precursor molecule enrichment values or asymptotic isotope enrichment of a fully turned over molecule of interest to represent the true precursor pool enrichment. Alternatively, the biosynthesis or breakdown rate may be calculated using an exponential decay curve by application of exponential or other die-away kinetic models (discussed further, infra).

a. Measuring Relative and Absolute Mass Isotopomer Abundances

Measured mass spectral peak heights, or alternatively, the areas under the peaks, may be expressed as ratios toward the parent (zero mass isotope) isotopomer. It is appreciated that any calculation means which provide relative and absolute values for the abundances of isotopomers in a sample may be used in describing such data, for the purposes of the present invention.

2. Calculating Labeled: Unlabeled Proportion of DNA Molecules of Interest

The proportion of labeled and unlabeled DNA molecules of interest is then calculated. The practitioner first determines measured excess molar ratios for isolated isotopomer species of a molecule. The practitioner then compares measured internal pattern of excess ratios to the theoretical patterns. Such theoretical patterns can be calculated using the binomial or multinomial distribution relationships as described in U.S. Pat. Nos. 5,338,686, 5,910,403, and 6,010,846, which are hereby incorporated by reference in their entirety. The calculations may include Mass Isotopomer Distribution Analysis (MIDA). Variations of Mass Isotopomer Distribution Analysis (MIDA) combinatorial algorithm are discussed in a number of different sources known to one skilled in the art. The method is further discussed by Hellerstein and Neese (1999), as well as Chinkes, et al. (1996), and Kelleher and Masterson (1992), and U.S. patent application Ser. No. 10/279,399, all of which are hereby incorporated by reference in their entirety.

In addition to the above-cited references, calculation software implementing the method is publicly available from Professor Marc Hellerstein, University of California, Berkeley.

The comparison of excess molar ratios to the theoretical patterns can be carried out using a table generated for a molecule of interest, or graphically, using determined relationships. From these comparisons, a value, such as the value p, is determined, which describes the probability of mass isotopic enrichment of a subunit in a precursor subunit pool. This enrichment is then used to determine a value, such as the value Aₓ*, which describes the enrichment of newly synthesized proteins for each mass isotopomer, to reveal the isotopomer excess ratio which would be expected to be present, if all isotopomers were newly synthesized.

Fractional abundances are then calculated. Fractional abundances of individual isotopes (for elements) or mass isotopomers (for molecules) are the fraction of the total abundance represented by that particular isotope or mass isotopomer. This is distinguished from relative abundance, wherein the most abundant species is given the value 100 and all other species are normalized relative to 100 and expressed as percent relative abundance. For a mass isotopomer Mₓ,

Fractional abundance of

$${M}_{x} = {A}_{x} = \frac{Abundance {M}_{x}}{{\sum }_{i = 0}^{n} Abundance {M}_{i}} ,$$

where 0 to n is the range of nominal masses relative to the lowest mass (M₀) mass isotopomer in which abundances occur.

Δ Fractional abundance (enrichment or depletion)=

$${( {A}_{x} )}_{e} - {( {A}_{x} )}_{b} = {( \frac{Abundance {M}_{x}}{{\sum }_{i = 0}^{n} Abundance {M}_{i}} )}_{e} - {( \frac{Abundance {M}_{x}}{{\sum }_{i = 0}^{n} Abundance {M}_{i}} )}_{b} ,$$

where subscript e refers to enriched and b refers to baseline or natural abundance.

In order to determine the fraction of polymers that were actually newly synthesized during a period of precursor administration, the measured excess molar ratio (EMₓ) is compared to the calculated enrichment value, Aₓ*, which describes the enrichment of newly synthesized biopolymers (e.g., DNA molecule) for each mass isotopomer, to reveal the isotopomer excess ratio which would be expected to be present, if all isotopomers were newly synthesized.

3. Calculating Molecular Flux Rates

The method of determining rate of synthesis includes calculating the proportion of mass isotopically labeled subunit present in the molecular precursor pool, and using this proportion to calculate an expected frequency of a molecule of interest containing at least one mass isotopically labeled subunit. This expected frequency is then compared to the actual, experimentally determined isotopomer frequency of the molecule of interest. From these values, the proportion of the DNA molecule of interest which is synthesized from added isotopically labeled precursors during a selected incorporation period can be determined. Thus, the rate of synthesis during such a time period is also determined.

A precursor-product relationship may then be applied. For the continuous labeling method, the isotopic enrichment is compared to asymptotic (i.e., maximal possible) enrichment and kinetic parameters (e.g., synthesis rates) are calculated from precursor-product equations. The fractional synthesis rate (kₛ) may be determined by applying the continuous labeling, precursor-product formula:

kₛ=[−ln(1−f)]/t,

where f=fractional synthesis=product enrichment/asymptotic precursor/enrichment

and t=time of label administration of contacting in the system studied.

For the discontinuous labeling method, the rate of decline in isotope enrichment is calculated and the kinetic parameters of the DNA molecules of interest are calculated from exponential decay equations. In practicing the method, biopolymers (e.g., DNA molecules) are enriched in mass isotopomers, preferably containing multiple mass isotopically labeled precursors. These higher mass isotopomers of the molecules of interest, e.g., molecules containing 3 or 4 mass isotopically labeled precursors, are formed in negligible amounts in the absence of exogenous precursor, due to the relatively low abundance of natural mass isotopically labeled precursor, but are formed in significant amounts during the period of molecular precursor incorporation. The molecules of interest taken from the cell, tissue, or organism at the sequential time points are analyzed by mass spectrometry, to determine the relative frequencies of a high mass isotopomer. Since the high mass isotopomer is synthesized almost exclusively before the first time point, its decay between the two time points provides a direct measure of the rate of decay of the DNA molecule of interest.

Preferably, the first time point is at least 2-3 hours after administration of precursor has ceased, depending on mode of administration, to ensure that the proportion of mass isotopically labeled subunit has decayed substantially from its highest level following precursor administration. In one embodiment, the following time points are typically 1-4 hours after the first time point, but this timing will depend upon the replacement rate of the biopolymer pool.

The rate of decay of the molecule of interest is determined from the decay curve for the three-isotope molecule of interest. In the present case, where the decay curve is defined by several time points, the decay kinetic can be determined by fitting the curve to an exponential decay curve, and from this, determining a decay constant.

Breakdown rate constants (k_{d}) may be calculated based on an exponential or other kinetic decay curve:

k_{d}=[−ln f]/t.

As described, the method can be used to determine subunit pool composition and rates of synthesis and decay for substantially any biopolymer which is formed from two or more identical subunits which can be mass isotopically labeled. Other well-known calculation techniques and experimental labeling or de-labeling approaches can be used (e.g., see Wolfe, R. R. Radioactive and Stable Isotope Tracers in Biomedicine: Principles and Practice of Kinetic Analysis. John Wiley & Sons; (March 1992)) for calculation flux rates of molecules and flux rates through metabolic pathways of interest (in the present invention, DNA synthesis and/or DNA degradation).

F. Uses of the Methods of the Present Invention

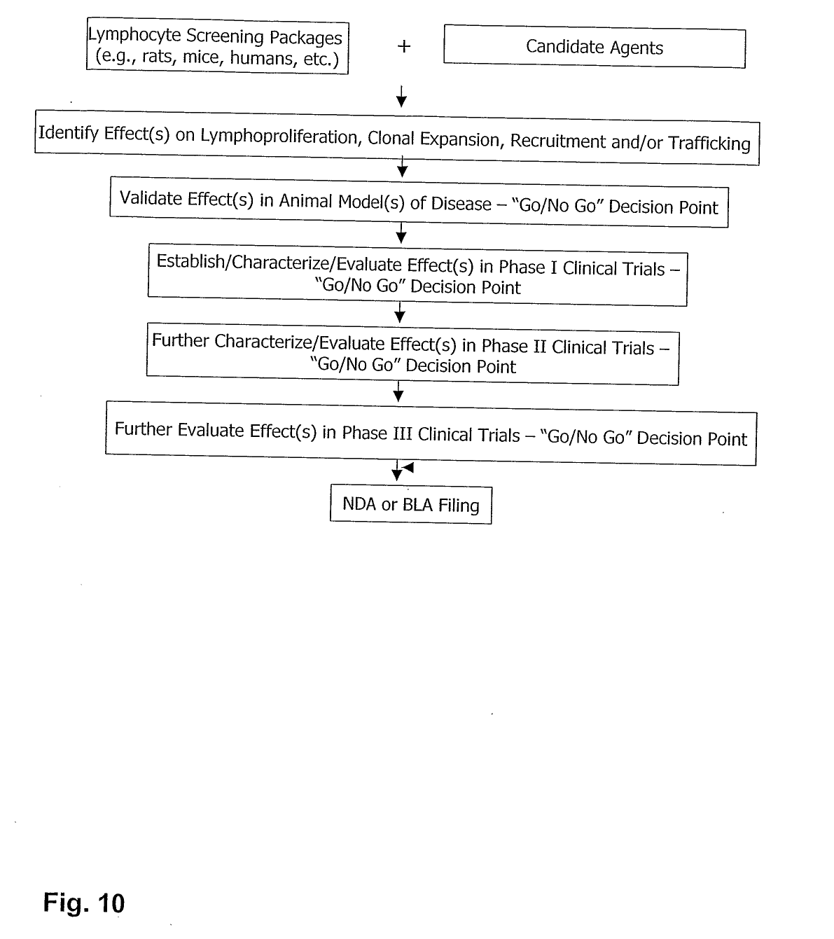

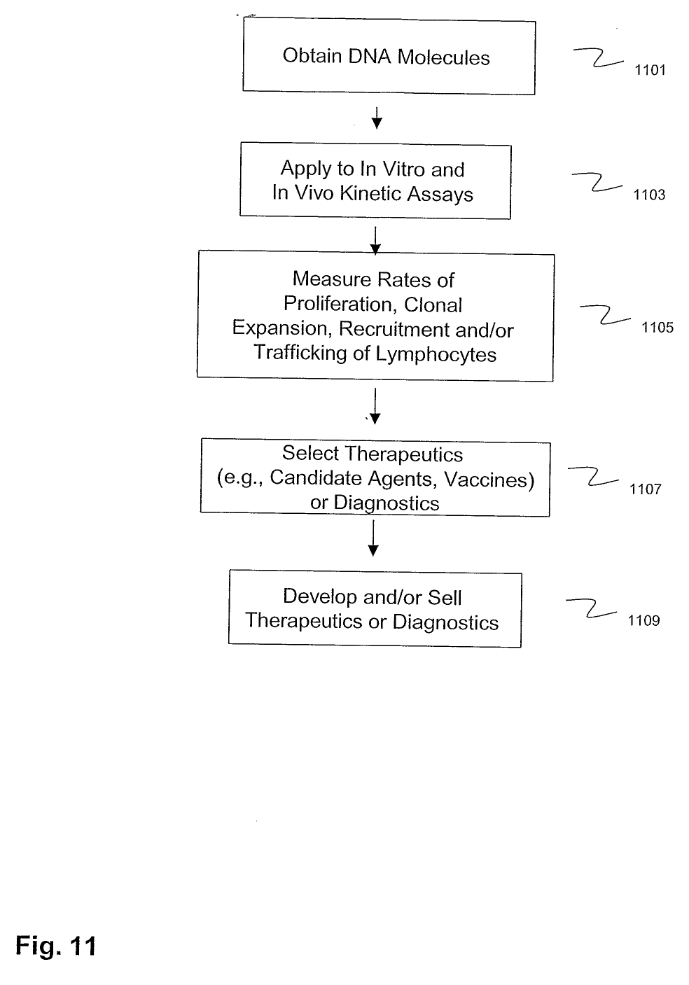

The methods disclosed herein find use in the drug discovery, development, and approval (DDDA) process (FIGS. 10-11). In particular, the methods of the present invention allow for, inter alia:

The in vivo measurement of lymphocyte proliferation (lymphoproliferation), clonal expansion, cell recruitment and/or trafficking in response to various external stimuli including vaccines, candidate agents, and environmental pollutants.

By “lymphoproliferation” herein is meant the reproduction of lymphocytes. Lymphoproliferation includes, but is not limited to, clonal expansion.

By “proliferation” herein is meant cell reproduction, during which one cell (the “parental” cell) divides to produce two daughter cells.

By “clonal expansion” here is meant the reproduction of lymphocytes from a single parent lymphocyte.

By “recruitment” herein is meant the relocation of lymphocytes near or to a site of interest, such as due to the presence of an antigen or the change in the concentration of cytokines. Of particular interest is recruitment to lympho nodes.

By “lymphocyte trafficking” herein is meant the migration of lymphocytes near or to a site of interest, such as an inflamatory site.

The invention finds use in methods to identify candidate agents that modulate lymphoproliferation, clonal expansion, cell recruitment and/or trafficking. Including within the term “modulate” are agents that reduce (e.g. one or more properties of lymphoproliferation, clonal expansion, cell recruitment and/or trafficking), or those that increase (eg.e activate) one or more properties of lymphoproliferation, clonal expansion, cell recruitment and/or trafficking. In general, “reduction” or “increase” is a change of at least about 10%, 20%, 30%, 40%, 50%, 60%, 70%, 80%, 90% or 100%, with higher increase being possible.

Optimization of antigen dose for vaccination, such as increase or decease the dosage of vaccine to achieve a desirable result;

Optimization of adjuvant for vaccination, such as increase or decease the dosage of adjuvant to achieve a desirable result;

Measurement of memory by label retention, such as by counting the amount of label isotope in isolated DNA or isolated cells;

Measurement of memory by recall after secondary immunization, such as, such as by counting the amount of label isotope in isolated DNA or isolated cells;

Determining therapeutic index in vivo;

The assessing of basal rates of lymphoproliferation, clonal expansion, cell recruitment and/or trafficking;

The quantitative comparisons of drugs, doses, and therapeutic regimes in vivo and in vitro;

A rapid, high-throughput, scaleable assay.

By “therapeutic index” (also known as “therapeutic ratio” or “margin of safety”) herein is meant a comparison of the amount of a therapeutic agent that causes the therapeutic effect to the amount that causes toxic effects. Quantitatively, it is the ratio given by the dose required to produce the toxic effect divided by the therapeutic dose. A commonly used measure of therapeutic index is the lethal dose of a drug for 50% of the population (LD₅₀) divided by the effective dose for 50% of the population (ED₅₀).

$$Therapeutic ratio = \frac{{LD}_{50}}{{ED}_{50}}$$

The methods described herein are applicable for screening candidate drug agents, FDA phase I and II human validation studies of candidate drug agents, FDA phase III approval of candidate drug agents, and FDA phase IV approval studies, or other post approval market positioning or mechanism of drug action studies.

In one embodiment, the methods allow for assessing effects on lymphoproliferation, clonal expansion, cell recruitment and/or trafficking to be observed after a living system is exposed to a candidate agent or a combination or mixture of candidate agents. The data generated and analyzed is therefore useful in the drug discovery, development, and approval (DDDA) process as it facilitates the DDDA decision-making process; i.e., it provides useful information for decision-makers in their decision to continue with further development of a candidate agent or a combination of candidate agents (e.g., if the inhibition or stimulation data concerning lymphoproliferation, clonal expansion, cell recruitment and/or trafficking appear promising) or to cease said efforts, for example, if the data concerning lymphoproliferation, clonal expansion, cell recruitment and/or trafficking appear unfavorable (see FIG. 10 for a graphical depiction of this process).

Moreover, the methods allow for the skilled artisan to identify, select, and/or characterize “best in breed” in a class of candidate agents (i.e., “best in class”). Once identified, selected, and/or characterized, the skilled artisan, based on the information generated by the methods of the present invention, can decide to evaluate the “best in breed” further or to license the candidate agent to another entity such as a pharmaceutical company or biotechnology company (see FIG. 11).

In another embodiment, the methods of the present invention allow for the characterization or evaluation (or both the characterization and evaluation) of toxic effects to tissues and cells from exposure to industrial chemicals, food additives, cosmetics, and environmental pollutants (e.g., an inhibition of lymphoproliferation, clonal expansion, cell recruitment and/or trafficking or, in some cases, a stimulation of lymphoproliferation, clonal expansion, cell recruitment and/or trafficking, from environmental exposure leading to a toxic injury including disease). The methods of the present invention can be used to establish programs to identify and explore the molecular mechanisms of industrial, food, cosmetic, and environmental toxicants on tissues and cells to further public health goals.

In one embodiment, data generated by the methods of the present invention may be relevant to understanding an underlying molecular pathogenesis, or causation of, one or more immunological diseases (e.g., lymphoproliferative disorders such as leukemias including chronic lymphocytic leukemias). In another embodiment, data generated by the methods of the present invention may shed light on fundamental aspects of the initiation, progression, severity, pathology, aggressiveness, grade, activity, disability, mortality, morbidity, disease sub-classification or other underlying pathogenic or pathologic feature of an immunological disease of interest.

In yet another embodiment, the data generated by the methods of the present invention may provide elucidation on fundamental aspects of the prognosis, survival, morbidity, mortality, stage, therapeutic response, symptomology, disability or other clinical factor of an immunological disease of interest, particularly of diseases associated with altered lymphocyte proliferation. Two or more biomarkers may be measured independently or concurrently (e.g., DNA synthesis and M protein turnover).

Known animal models of disease may be used as part of the present invention. Such animal models of disease may include, but are not limited to, immunological models of disease such as lymphocytic leukemias, lymphomas, inflammatory diseases, HIV/AIDS and the like.

In another embodiment, the methods of the invention are useful in detecting toxic effects of candidate agents such as industrial or occupational chemicals, food additives, cosmetics, or environmental pollutants/contaminants on living systems such as tissues or cells. Toxicity in the context of the present invention is usually measured by an undesirable alteration in lymphoproliferation, clonal expansion, cell recruitment and/or trafficking. As outlined herein, the alteration can either be stimulation or inhibition depending on the experimental context and chosen observable outcome. In some embodiments, toxic effects may include end-organ toxicity. End-organ toxicity may include, but is not limited to, lymphoproliferation, clonal expansion, cell recruitment and/or trafficking in primary and secondary lymphoid organs.

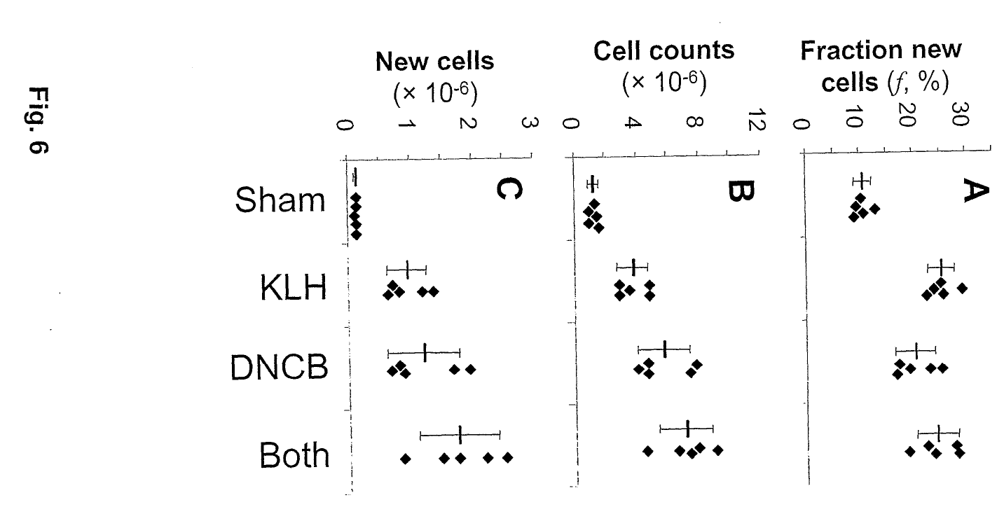

FIG. 11 illustrates the use of the inventions herein in a drug discovery process. At step 01 a plurality of candidate agents are selected. At step 03 the flux rate of DNA synthesis is studied within lymphocytes, preferably according to the methods discussed herein. In alternative embodiments, step 03 is conducted first when the inventions are used, for example, in a target discovery process. At step 05 relevant flux rates are identified. For example, if it is desirable to reduce the flux rate of a particular biomarker of cell proliferation in a particular phenotypic state, a compound that reduces that flux rate will be considered generally more useful, and conversely a compound that increases that flux rate will be considered generally less desirable (e.g., de novo DNA synthesis). In a target discovery process, a particular phenotype that has increased or decreased flux rates with respect to another phenotype (e.g., diseased vs. not diseased) may be considered a good therapeutic or diagnostic target or in the pathway of a good therapeutic or diagnostic target. At step 07 candidate agents of interest and vaccines of interest (collectively, “therapeutics”), targets of interest, or diagnostics are selected and further used and further developed. In the case of targets, such targets may be the subject of, for example, well known small molecule screening processes (e.g., high-throughput screening of candidate agents such as new chemical entities or others) and the like. At step 09 the candidate agents, vaccines, or diagnostics are sold or distributed. What is sold or distributed may be “best in breed,” so identified by the methods of the present invention. It is recognized of course that one or more of the steps in the process in FIG. 6 will be repeated many times in most cases for optimal results.

In yet a further embodiment, the methods of the present invention can be used to discover candidate agents that have immunosuppressive effects. Certain progressive immunodeficiency syndromes, including HIV-1 infection and acquired immunodeficiency syndrome (AIDS), are characterized by chronic immune activation. This activation is believed to contribute to the disease progression (inexorable loss of immune function) by several mechanisms. Use of classic immunosuppressive agents is problematic, however, in these settings because of concerns about worsening the underlying immunodeficiency present. One embodiment of the invention comprises a drug discovery tool and a therapeutic strategy using drugs so discovered. Applicants first disclose herein a means of identifying candidate agents that interfere with lymphocyte trafficking and recruitment into lymph nodes (LN) and for identifying optimal doses or regimens of such agents. Examples of candidate agents discovered by this method are disclosed.

In yet another embodiment, Applicants disclose a therapeutic method for slowing the progression of immune deficiency in certain diseases of immune deficiency, by reducing chronic immune activation without cytotoxic activity or other classic immune toxicities. The method consists of administering agents that reduce or prevent the homing of lymphocytes to inductive sites (such as LNs) in the gut or peripheral tissues, or both, to a subject with an incipient or existing immune deficiency syndrome. As a consequence, several processes involved in the progressive loss of immune function can be reduced, including: lymphocyte activation and proliferation; secondary depletion of naïve T-cells; loss of antigen-specific repertoire in the memory/effector T-cell pool; altered lymphocyte homeostasis, including thymic dysfunction; fueling of HIV-1 replication, in HIV-1 infection, by provision of target cells; and damage to lymph node architecture, including fibrosis. Agents that interfere with lymphocyte trafficking need not have cytotoxic actions and are therefore ideal therapeutic candidates for use in immunodeficiency syndromes.

G. Isotopically-Perturbed Molecules

In another variation, the methods provide for the production of isotopically-perturbed molecules (e.g., nucleic acids). These isotopically-perturbed molecules comprise information useful in determining alterations in lymphoproliferation. Once isolated from a lymphocyte and/or a tissue of an organism, one or more isotopically-perturbed molecules are analyzed to extract information as described, supra.

H. Kits

The invention also provides kits for measuring changes in lymphoproliferation. The kits may include isotope-labeled precursor molecules, and may additionally include chemical compounds known in the art for separating, purifying, or isolating proteins, and/or chemicals necessary to obtain a tissue sample, automated calculation software for combinatorial analysis, and instructions for use of the kit.

Other kit components, such as tools for administration of water (e.g., measuring cup, needles, syringes, pipettes, IV tubing), may optionally be provided in the kit. Similarly, instruments for obtaining samples from the cell, tissue, or organism (e.g., specimen cups, needles, syringes, and tissue sampling devices) may also be optionally provided.

I. Information Storage Devices

The invention also provides for information storage devices such as paper reports or data storage devices comprising data collected from the methods of the present invention. An information storage device includes, but is not limited to, written reports on paper or similar tangible medium, written reports on plastic transparency sheets or microfiche, and data stored on optical or magnetic media (e.g., compact discs, digital video discs, optical discs, magnetic discs, and the like), or computers storing the information whether temporarily or permanently. The data may be at least partially contained within a computer and may be in the form of an electronic mail message or attached to an electronic mail message as a separate electronic file. The data within the information storage devices may be “raw” (i.e., collected but unanalyzed), partially analyzed, or completely analyzed. Data analysis may be by way of computer or some other automated device or may be done manually. The information storage device may be used to download the data onto a separate data storage system (e.g., computer, hand-held computer, and the like) for further analysis or for display or both. Alternatively, the data within the information storage device may be printed onto paper, plastic transparency sheets, or other similar tangible medium for further analysis or for display or both.

### EXAMPLES

The following non-limiting examples further illustrate the invention disclosed herein:

### Example 1

### Protocols

The protocols in this Example 1 were followed for all experiments described below.

Reagents. Keyhole limpet hemocyanin (KLH) and all xenobiotics such as cyclosporine A and the others described below were from Sigma (St. Louis, Mo.) or from other well known commercial vendors of chemicals. Heavy water was from Cambridge Isotope Labs (Cambridge, Mass.). Tissue culture grade, endotoxin-free sterile PBS from Cellgro (City/State) was used for sham immunizations and as a diluent for KLH, which was stored in aliquots at −20° C. All other chemicals were from Sigma unless mentioned otherwise.

Animals. All animal procedures received prior written approval by KineMed's Animal Care and Use Committee. Animals (female C57BI/6 and Balb/c mice, unless mentioned otherwise) were housed at KineMed's animal facility under specific pathogen-free conditions, subject to a 12 h light/12 h dark cycle, and given water and standard chow ad libitum. They were purchased at 6 weeks of age and acclimatized on site for at 2-7 days prior to immunization.

PLNA and heavy water labeling. The kinetic PLNA was performed as follows, except as noted in the Figure legends. In all experiments, groups of n=4 or 5 Balb/c mice were immunized subcutaneously on day 0 in the left hind footpad (toe to heel direction) with 20 μl PBS or drug vehicle (negative control group), or with 20 μl PBS containing 5-25 μg KLH (positive control group). Heavy water labeling was initiated on day 0 by intraperitoneal administration of 99.9% ²H₂O containing 0.9% w/v NaCl at 35 ml/kg body weight, and animals were maintained on 8% ²H₂O in drinking water for the remainder of the study. At day 7 post immunization, animals were bled into heparinized tubes by cardiac puncture under isoflurane anesthesia and sacrificed by cervical dislocation. The blood was centrifuged at 1500×g for 10 minutes, and heparinized plasma was stored at −20° C. for determination of body water ²H enrichment; a second aliquot was frozen for serologic studies. Bone marrow was harvested from the right hindlimb femur by flushing the bone with 1 ml PBS using a 25 ga cannula and centrifuging the tissue and cell suspension at 450×g for 5 min.

Analysis of PLN cells. Draining PLN were dissected from the left hind limb, mechanically dispersed, using tweezers, into ice-cold RPMI 1640 containing 10% fetal bovine serum and antibiotics. The cell suspensions were thoroughly mixed and strained through a 35 μm cell strainer fitted to a 12×75 mm polystyrene tube (BD Biosciences, San Jose, Calif.) and centrifuged at 450×g at 4° C. for 5 min. Cells were suspended in 100 μl staining buffer (PBS containing 0.5% w/v bovine serum albumin, 2 mM EDTA, and 0.05% sodium azide), blocked by addition of 20 μl mg/ml murine IgG (Sigma) and 2 μl anti-CD16/CD32 mAb (Miltenyi Biotec, City/State) at 4° C. for 15 min., and stained with anti-CD3-FITC and anti-B220-PE/Cy₅ (eBioscience, 1 μl each) for 15 min. The samples were diluted to 900 μl with staining buffer, mixed thoroughly with 100 μl Flow-Count fluorospheres, and analyzed on an Epics XL flow cytometer (Beckman Coulter, City/State) for determination of absolute lymph node cell counts and percentages and absolute counts of T cells, B cells, and non-T/B cells. Absolute cell counts per lymph node were determined as:

Cell count=(number of cell events/number of bead events)×bead concentration per μl×100 μl.

Contralateral (right) PLN of control mice were used for single-stain and isotype controls. Cells were identified by forward and side scatter with exclusion of doublets based on plots of forward scatter pulse height vs. area. An aliquot was pelleted at 450×g for 5 min. for DNA extraction. In some experiments, the remainder of the cells was washed in PBS, fixed in 2% w/v paraformaldehyde in PBS, and T and B cells were sorted on a Coulter Epics Elite cell sorter. Sorted cells were routinely >99% pure upon reanalysis.

Analysis of ²H incorporation into purine dR. DNA was extracted from samples of bone marrow, total PLN cells, or sorted T and B cells using DNEasy kits (Qiagen, Valencia, Calif.) according to the manufacturer's instructions. Aliquots (0.5 μg) were hydrolyzed to deoxyribonucleotides by overnight incubation at 37° C. with 0.5 units nuclease SI (Sigma) and 0.25 units potato acid phosphatase (Calbiochem) in 75 mM sodium acetate buffer, pH 4.8, containing 0.15 mM ZnSO₄. Deoxyribose was released selectively from purine deoxyribonucleotides by treatment with acetic acid at 100° C. for 30 min., and its aldehyde group was simultaneously derivatized with pentafluorobenzyl hydroxylamine. The hydroxyl groups were acetylated using acetic anhydride and N-methylimidazole, and the resultant pentafluorobenzyl tetraacetate derivative was extracted into dichloromethane, dried under vacuum, and redissolved in 100 μl ethyl acetate. The derivative was analyzed by gas chromatography/mass spectrometry (GC/MS) as described supra, with selected ion monitoring at m/z 435 (for the parent mass isotopomer, M0) and 436 (M1). After integration of the M₀ and M₁ peak area using ChemStation software, the excess mole fraction of the M1 mass isotopomer (EM1) was calculated as:

EM₁=[(M₁)/(M₀+M₁)]sample−[(M₁)/(M₀+M₁)]baseline.

The baseline mass isotopomer distribution was calculated from analyses of unlabeled deoxyribose or calf thymus DNA standards, which were matched to samples for M₀ abundance. Duplicate EM₁ values were routinely within 0.1% of the mean.

²H enrichment in body water was measured as described supra. Briefly, water was isolated from (50 μl) serum by microscale distillation and reacted with calcium carbide to transfer hydrogen atoms to acetylene. Acetylene was analyzed on a Series 3000 cycloidal mass spectrometer (Monitor Instruments, Cheswick, Pa.), and the abundance of the m/z=26 (M0) and 27 (M1) was compared to a standard curve prepared from known mixtures of ²H₂O and ¹H₂O. Body water ²H enrichments were 5% in animals maintained on 8% ²H₂O in drinking water. The mole fraction of ²H in body water was converted to a predicted EM, value for purine deoxyribose in fully turned-over DNA, using MIDA algorithms. When compared to this standard, bone marrow DNA in animals labeled for 24 days was 85-95% turned over; the balance likely reflects a small contribution by nucleotide salvage.

Calculations and statistics. Fractional turnover of PLN cell populations (f, the fraction of total, T, or B cells that have divided during the labeling period, representing clonal expansion, FIG. 1) was calculated as:

f=EM₁observed/EM₁maximal,

where EM₁maximal was represented, in most experiments, by bone marrow DNA from the same animal, or assumed to equal 0.14. Alternatively, for mice labeled for less than four days, EM₁maximal was represented by 0.9× the EMI value predicted from the animal's body water ²H enrichment (the factor of 0.9 represents dilution by salvage). The number of cells that have divided during the labeling period was calculated from the fractional turnover of the cells of interest and the absolute number of cells of interest per lymph node:

New cells=f×cell number.

Depending on the experimental design, groups of animals were compared by unpaired Student's t test, One-way or two-way ANOVA, as implemented in SigmaStat version 8.0. P values below 0.05 were considered significant.

### Example 2

### The Increase in PLN Cell f after Immunization is Antigen-Dependent

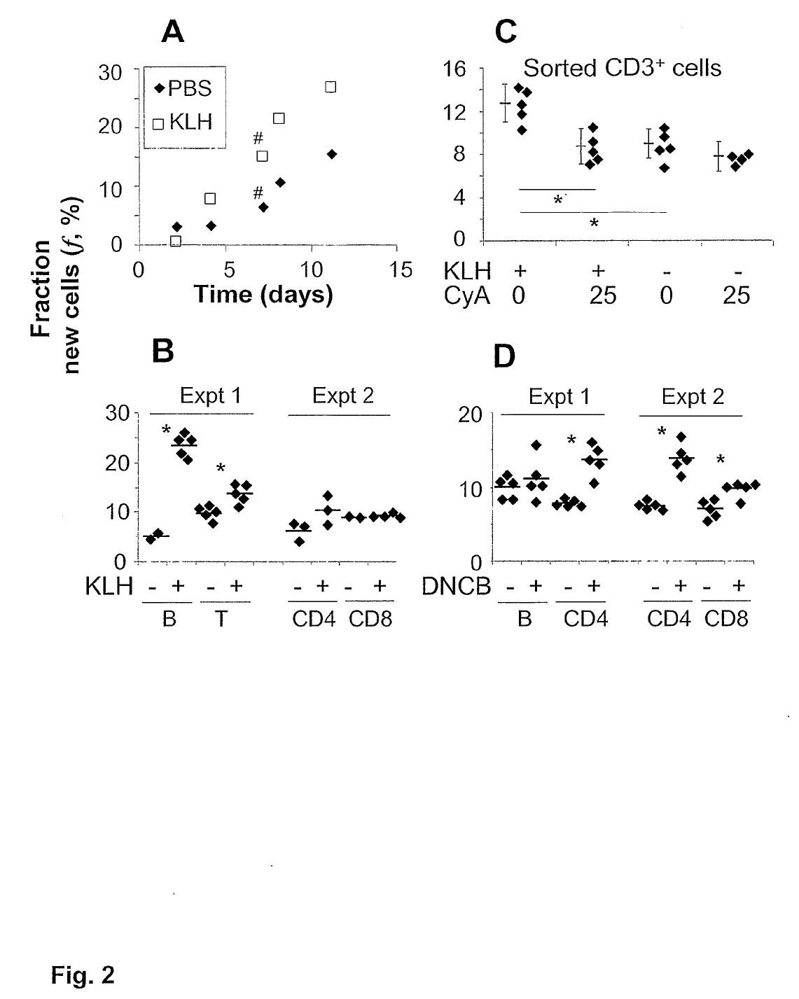

We next examined whether the increased f in PLN reflects antigen-dependent lymphoproliferation or preferential recruitment of proliferating over resting cells. First, we measured f in lymphocyte subsets (FIG. 2B). A pronounced increase in f was found in PLN B cells from KLH-immune mice. A smaller increase in f was detected in total and CD4⁺ T cells, but not in CD8⁺ T cells (FIG. 2B), consistent with the known ability of the former, but not the latter, to be primed by exogenous proteins. Moreover, this result suggests that proliferation of B and CD4⁺ T cells is not due to LPS contamination, because even low doses of LPS increase the percentage of proliferating CD8⁺ T cells.

Second, the KLH-stimulated increase in f of T cells was inhibited by cyclosporin A (FIG. 2C), a well known calcineurin antagonist that prevents nuclear translocation of NFATc after antigenic stimulation. By contrast, baseline f of T cells was not significantly reduced by cyclosporin (FIG. 2C). Thus, calcineurin-dependent processes do not contribute much to baseline f. The cyclosporin A-sensitive component of the KLH-stimulated increase in T-cell f was probably due to antigen-driven proliferation.

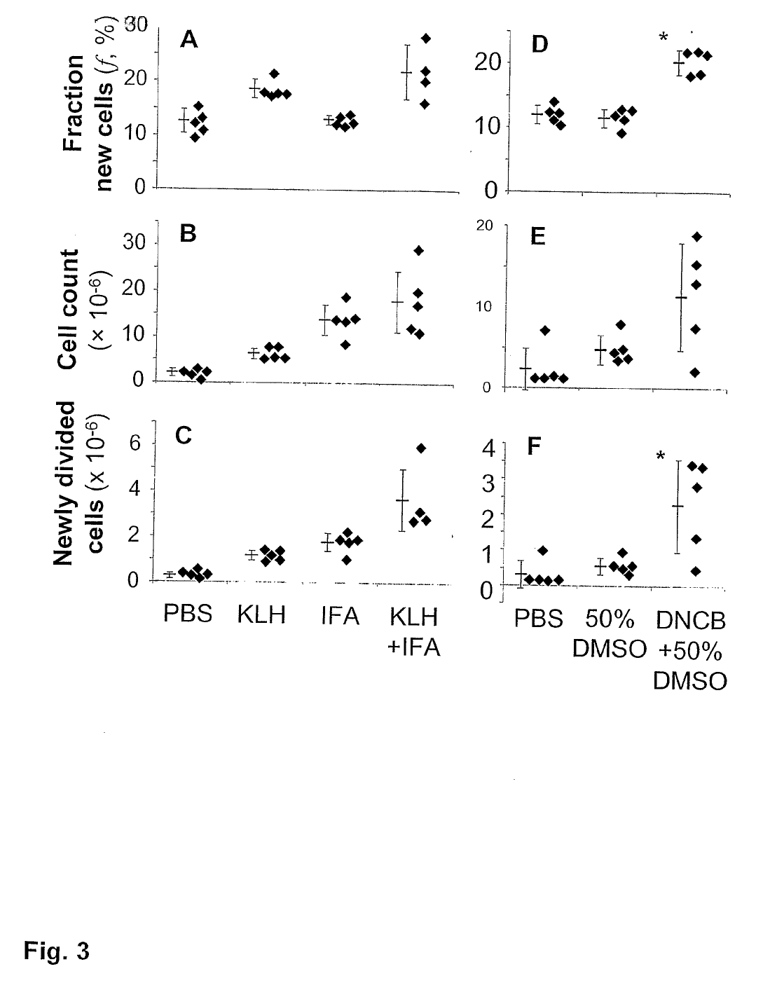

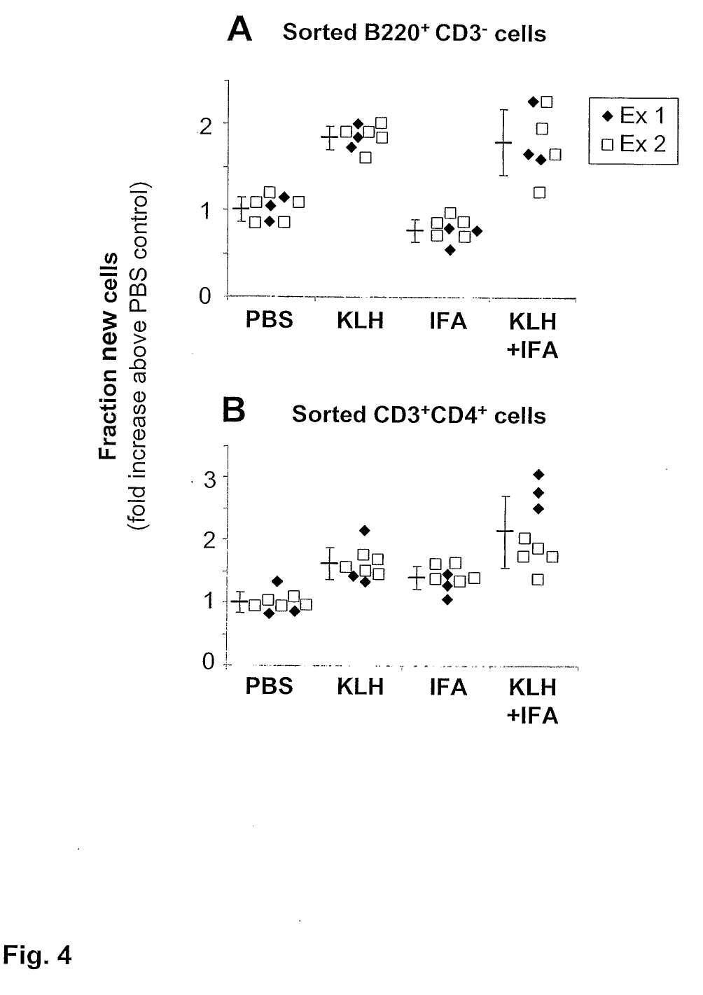

Third, we saw no marked changes in PLN cell f after recruiting cells to draining LN through s.c. injection of IFA (mineral oil) without antigen (FIG. 3A; “IFA” vs. “PBS”). PLN cell counts were increased, however (FIG. 3B); the absolute number of proliferating cells was increased accordingly (FIG. 3C). Thus, lymphocyte recruitment to PLN by IFA attracts resting and proliferating cells similarly (FIG. 1A, a vs. c). In B cells, f was decreased by IFA, and f of CD4⁺ T cells was increased, but these changes were slight (FIG. 4).

Fourth, PLN cell counts and f were increased after immunization of Balb/c mice with 2,4-dinitrochlorobenzene (DNCB), a low molecular weight allergen (FIG. 3D-F). The DMSO vehicle caused lymphocyte recruitment to the PLN (FIG. 3E), but did not increase f (FIG. 3D). DNCB stimulated f of sorted CD4⁺ T cells and CD8⁺ T cells (FIG. 2D); stimulation of B-cell f was variable, but low (FIG. 2D).

In summary, increased f in draining LN after protein or hapten immunization is primarily due to antigen-stimulated proliferation, and thus, represents clonal expansion. In contrast, lymphocyte recruitment to LN by nonspecific inflammatory stimuli increases cellularity and the absolute number of recently divided cells, but does not markedly change f.

### Example 3

### Differential Contributions from f (Clonal Expansion) and Cell Number to Lymphoproliferation

Absolute lymphoproliferation, i.e., the total number of LN cells that have divided during a period of ²H₂O exposure, can be calculated as (LN cellularity×f). By this definition, absolute lymphoproliferation equals the net accrual of divided cells in the LN, due to constitutive cell proliferation and death, cell recruitment to the LN, local antigen-driven proliferation, and activation-induced death.

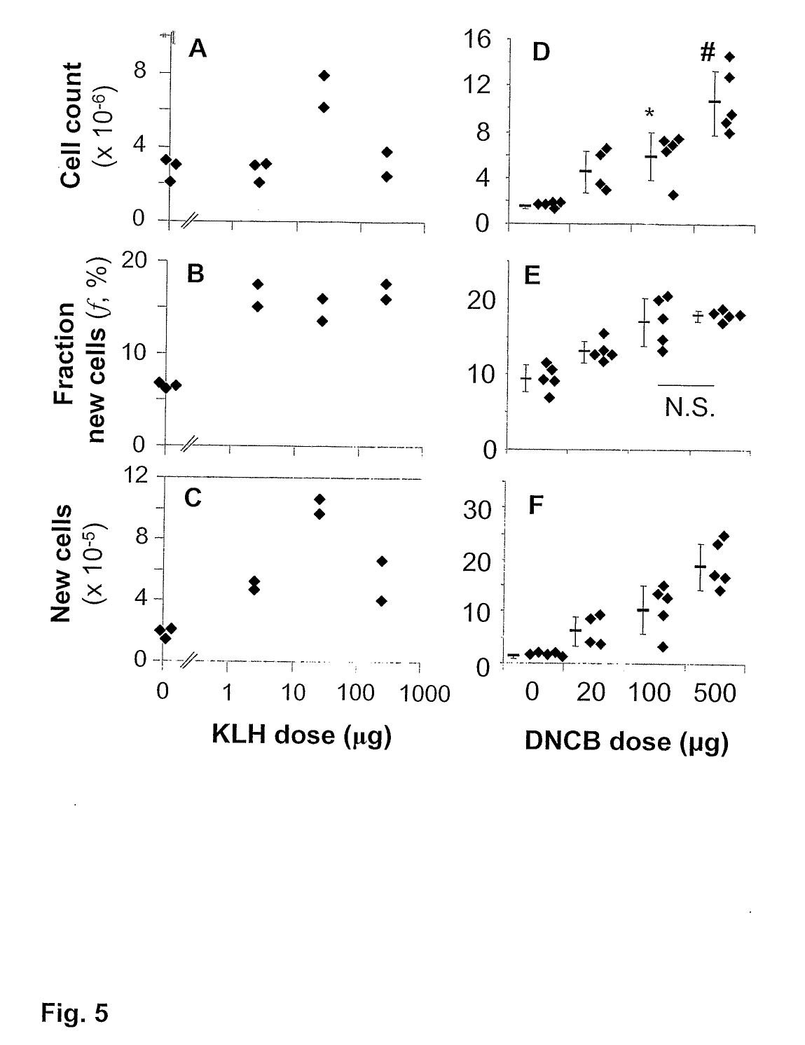

We determined the contributions from cellularity and f to absolute lymphoproliferation in draining LN 7 days after immunization of Balb/c mice with different doses of KLH (FIG. 5A-C). Cellularity was maximal around 25 μg KLH (FIG. 5A); a tenfold lower or higher antigen dose did not increase cellularity, compared to negative controls. In contrast, f was elevated to a dose-independent plateau over a 100-fold range of KLH doses (FIG. 5B). At KLH doses up to 2.5 μg, absolute lymphoproliferation (FIG. 5C) was driven by increased f without cell recruitment. Indeed, f was maximally increased after immunization with as little as 600 ng KLH (FIG. S3). Between 2.5 and 25 μg KLH, the further increase in absolute lymphoproliferation was driven by changes in cellularity, as was the decrease at 250 μg (FIG. 5A-C). Thus, the KLH-stimulated increase in f behaved like an on/off switch, with maximal effect at low antigen doses, and followed a different dose response than LN cellularity. Proliferating cells never comprised more than ≈20% of cells in the PLN, even at antigen doses that increased PLN cellularity by 2- to 3-fold.

Somewhat different results were obtained after immunization with DNCB (FIG. 5D-F). Both f and absolute lymphoproliferation were sub-maximal at low doses, but f reached a plateau at doses above 100 μg DNCB. The further increase in absolute lymphoproliferation at higher doses was driven by increased cellularity. Thus, differential dose-response relationships for LN cellularity and f (clonal expansion) were also observed with DNCB at high doses.

To test whether the plateau of f (clonal expansion) after KLH immunization reflected the lack of an antigen depot or of inflammatory stimuli, we immunized animals with KLH emulsified in IFA (33) (FIG. 3A-C). Strikingly, the increase in PLN cell f 7 days after immunization with KLH in IFA was indistinguishable from the KLH effect without adjuvant (FIG. 3A). Nor did IFA augment the KLH effect on f at day 4 or day 10 post immunization. Similarly, the KLH effect on B cell and CD4⁺ T-cell f was not markedly augmented by IFA (FIG. 4). IFA greatly increased absolute lymphoproliferation, both in the presence and absence of KLH (FIG. 3C), due to increased cellularity in draining LN (FIG. 3B). Thus, the lack of adjuvanticity with KLH alone did not explain the plateau in f. Moreover, the increased absolute lymphoproliferation in PLN of IFA-treated animals, with or without KLH, was driven primarily by increased lymphocyte recruitment.

### Example 4

### Relationship Between the Plateau Value of f (Clonal Expansion) and Precursor Frequency

To test whether the plateau in f was set by precursor frequency, we performed sequential immunizations with KLH and DNCB (FIG. 6). The responder cell populations for these unrelated antigens should be derived from different antigen-specific precursors. We reasoned that if the plateau level of f for each antigen is related to the frequency of cognate precursors, the increase in f after immunization with both antigens should be the sum of the increments seen with each antigen alone. Surprisingly, after immunization with optimal doses of DNCB and KLH, f in total PLN cells was not increased above the value obtained with KLH alone (FIG. 6A). This was not due to antigenic competition: absolute lymphoproliferation and cellularity were higher after immunization with both antigens than with either antigen alone (FIGS. 6B, C).

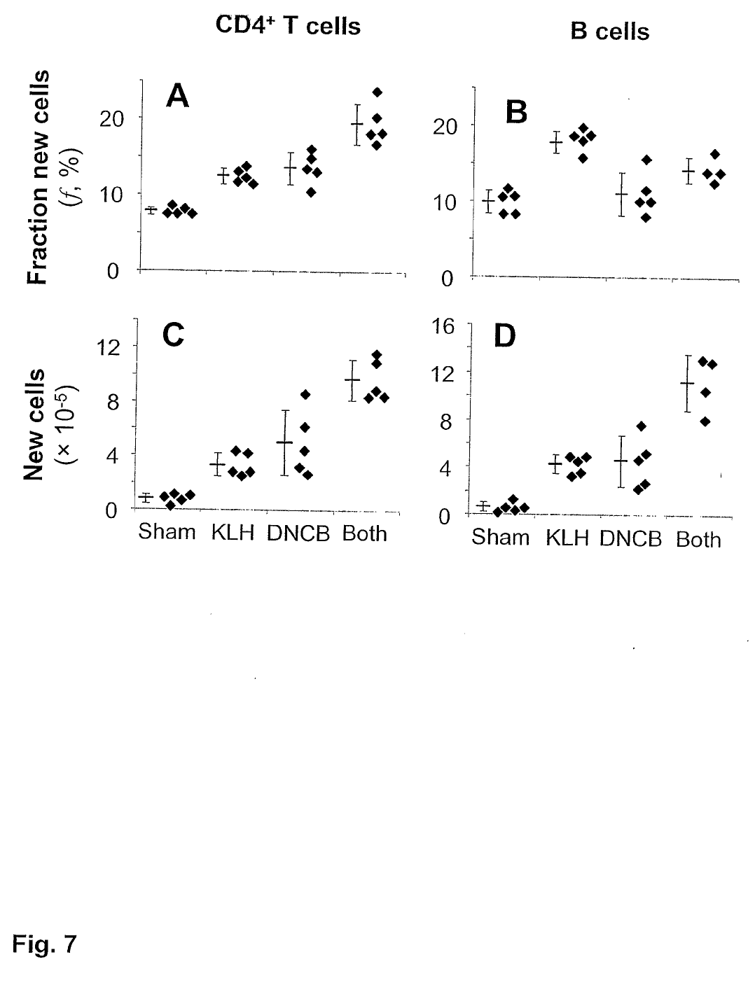

Dual immunization exerted different effects on f in B and CD4+ T cells, explaining the unexpected behavior of total PLN cell f (FIG. 7). In CD4+ T cells, f after immunization with both KLH and DNCB was greater than with either antigen alone (FIG. 7A). By this criterion, precursor frequency was limiting for CD4+ T-cell f. Non-additive behavior was, however, observed for B cells: f after immunization with both antigens was intermediate between values for either antigen alone (FIG. 7B). Thus, B-cell f at optimal antigen doses was not limited by precursor frequency. The plateau in f seen for total lymphocytes (FIG. 6A) is therefore an emergent property of the assembled system. Absolute lymphoproliferation in both subsets was approximately additive (FIGS. 6C, D).

### Example 5

### Differential Drug Effects on f (Clonal Expansion) and Cellularity

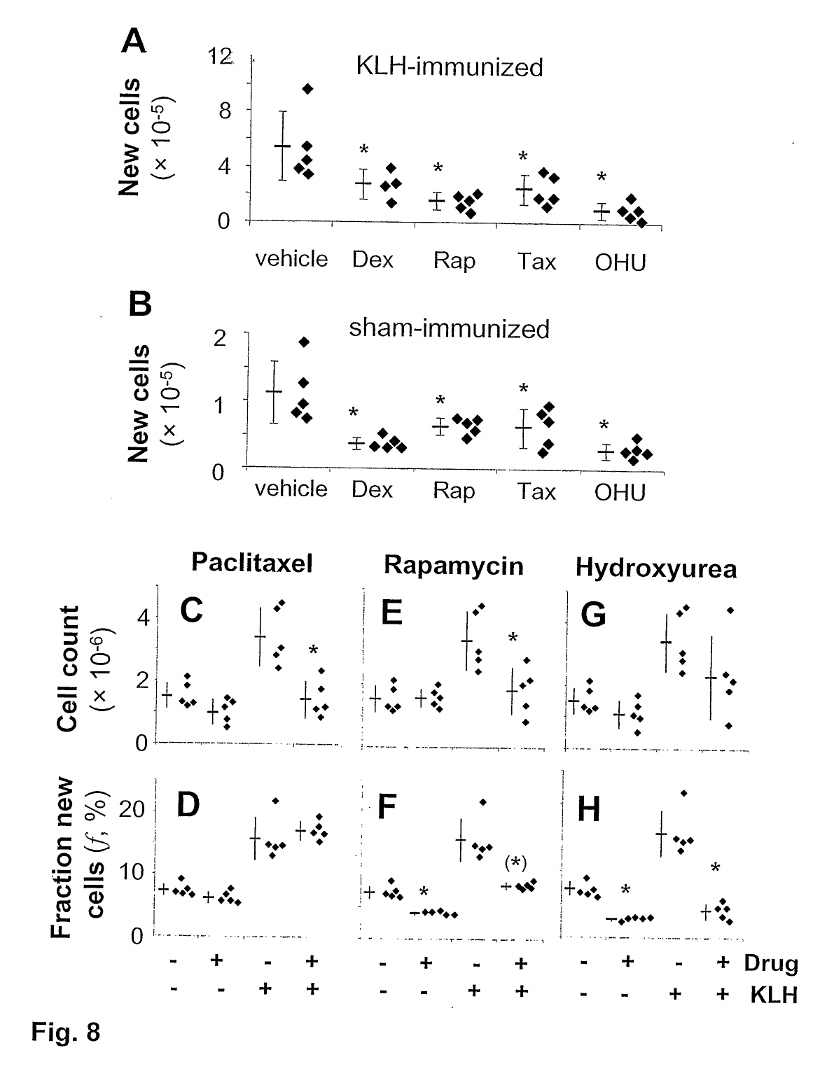

If LN cellularity and f (clonal expansion) are differentially regulated during immune responses, they might also be independently affected by antiproliferative or immunomodulatory drugs. Accordingly, we examined drug effects at doses chosen to give comparable effects on absolute lymphoproliferation (FIGS. 8A, B). Drugs differed in their effects on cellularity and f (FIG. 8C-H). For instance, rapamycin inhibited f of total PLN cells, both at baseline and after KLH stimulation; cellularity was reduced after only antigenic stimulation. The ribonucleotide reductase inhibitor, hydroxyurea, strongly inhibited both baseline and antigen-stimulated f, with modest and variable effects on cellularity. The microtubule-stabilizing agent, paclitaxel, reduced PLN cellularity but, surprisingly, did not affect f, even though at the doses used (10 mg/kg/d, i.p.), we previously saw strong inhibition of f of xenografted human tumor cells in mice. Dexamethasone also reduced cellularity more than f. The drugs may differentially affect homeostatic turnover, clonal expansion, and net cell recruitment. Moreover, they may kill resting as well as proliferating lymphocytes, in unknown proportions, as is probably the case for dexamethasone. These effects may be difficult to dissect, but the data show that drugs acting on different molecular targets exert divergent effects on f (clonal expansion) and cellularity, confirming that these parameters are regulated independently.

### Example 6

### Setpoints for f are Strain Dependent

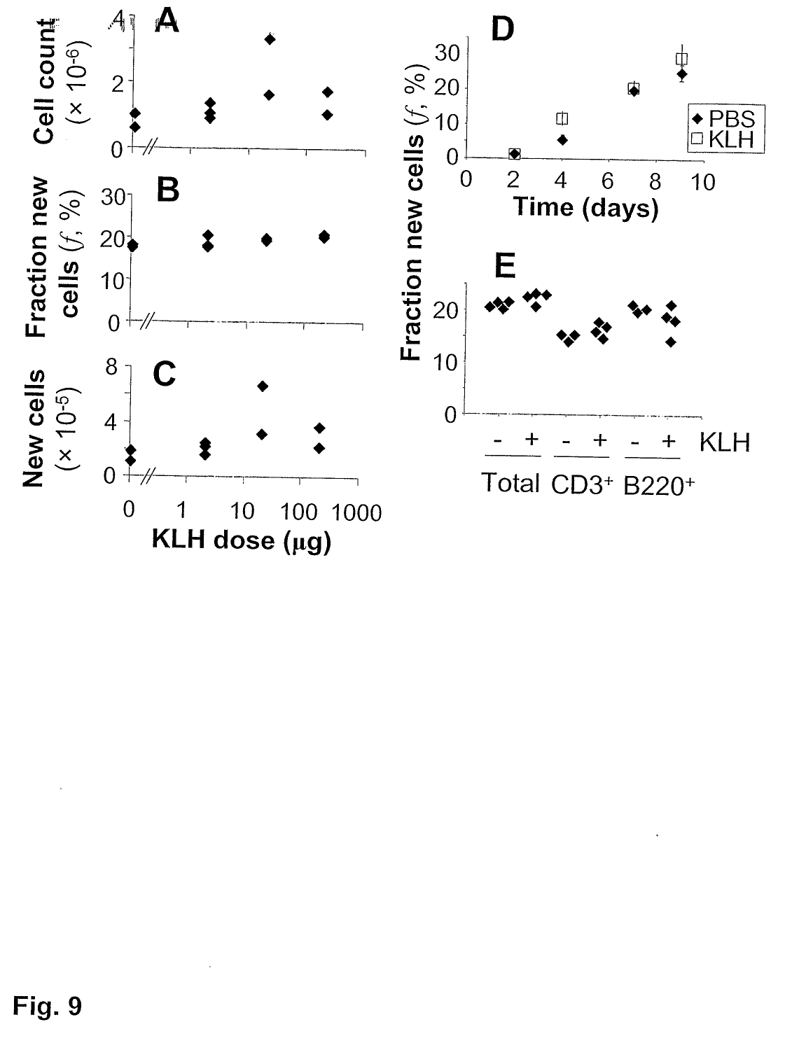

Finally, we examined whether the dissociation between f (clonal expansion) and LN cellularity after immunization was unique to the Balb/c strain. Cellularity in PLN of C57BI/6 (B6) mice 7 days after KLH immunization was dose-dependent, with a similar profile as in Balb/c mice (FIG. 9A). Baseline turnover of PLN cells in B6 mice was higher than in Balb/c (cf. FIGS. 9B and 5B). At day 7, KLH immunization of B6 mice stimulated no significant increase in f at any dose (FIG. 9B). The dose dependence of absolute lymphoproliferation in PLN was driven entirely by changes in cellularity (FIG. 9C). In a time course, a KLH-dependent, transient increase in f was seen at day 4, but not earlier or later (FIG. 9D). Neither B cells nor T cells showed a substantial increase in f at day 7 (FIG. 9E). Thus, the setpoints for baseline and KLH-stimulated f are strain-dependent, and thus, by inference, under genetic control.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety for all purposes to the same extent as if each individual publication, patent, or patent application were specifically and individually indicated to be so incorporated by reference.

We claim:

1. A method for evaluating the effect of a candidate agent on a lymphocyte population of a test vertebrate system, said method comprising:
a) exposing a test vertebrate system to at least one candidate agent;
b) exposing said test vertebrate system to at least one antigen;
c) administering one or more isotope-labeled substrates to said test system for a period of time sufficient for said isotope-labeled substrates to enter into DNA during replication;
d) obtaining a first sample comprising first lymphocytes from said test vertebrate system;
e) quantifying the isotopic enrichment of said first lymphocytes from said first sample;
f) providing the isotopic enrichment of control lymphocytes from a control vertebrate system;
g) comparing the ratio of enrichment in said first lymphocytes to the ratio of enrichment of said control lymphocytes; and
h) determining the effect of said agent on said first lymphocytes.

2. A method according to claim 1 wherein said first sample comprises at least one lymph node.

3. A method according to claim 1 or 2 wherein said determining step comprises measuring cellularity of said first lymphocytes from said first sample.

4. A method according to claim 1 or 2 wherein said determining step comprises measuring proliferation of said first lymphocytes from said first sample.

5. A method according to any of claims 1-3 further comprising measuring clonal expansion of said first lymphocytes from said first sample.

6. A method according to any of claims 1-5 further comprising measuring recruitment rates of said first lymphocytes into said first sample.

7. A method according to any of claims 1-6 further comprising measuring lymphocyte trafficking of said first lymphocytes into said first sample.

8. A method for measuring the generation of long-lived memory lymphocyte population in a test vertebrate, said method comprising:
a) exposing said test vertebrate system to at least one antigen;
b) administering one or more isotope-labeled substrates to said living system for a first period of time sufficient for said isotope-labeled substrates to enter into DNA during replication;
c) ceasing administration of said isotope-labeled substrate for a second period of time;
d) obtaining a first sample comprising first lymphocytes from said test vertebrate system; and
e) quantifying isotope label retention in DNA isolated from said first lymphocytes to calculate the generation of long-lived memory lymphocytes.

9. A method according to claim 8 wherein said test vertebrate system is exposed to a plurality of antigens.

10. A method according to claim 8 wherein said test vertebrate system is exposed to a plurality of antigen preparations, and said method further comprises comparing the number of long-lived memory lymphocytes.

11. A method according to claim 8 wherein said test vertebrate system is exposed to a plurality of antigen preparations, and said method further comprises comparing the life-span of long-lived memory lymphocytes.

12. A method according to any of claims 8-11 wherein said test vertebrate system is further exposed to at least one adjuvant.

13. A method according to any of claims 8-12 wherein said test vertebrate system is further exposed to a plurality of adjuvants.

14. A method according to claim 13 wherein the ability of at least one of said adjuvants is evaluated for the ability to enhance lymphoproliferation.

15. A method according to claim 13 wherein the ability of at least one of said adjuvants is evaluated for the ability to form stable memory cells.
